(19) United States
(12) Patent Application Publication
(10) Pub. No.: US 2024/0013064 A1
(43) Pub. Date: Jan. 11, 2024

(54) MACHINE LEARNING TECHNIQUES USING MODEL DEFICIENCY DATA OBJECTS FOR TENSOR-BASED GRAPH PROCESSING MODELS

(71) Applicant: Optum, Inc., Minnetonka, MN (US)

(72) Inventors: Paul J. Godden, London (GB); Erik A. Nystrom, Durham, NC (US); Gregory J. Boss, Saginaw, MI (US)

(21) Appl. No.: 17/811,229

(22) Filed: Jul. 7, 2022

(51) Int. Cl. G06N 3/12

(52) U.S. Cl. CPC G06N 3/126

(57) ABSTRACT

Various embodiments of the present invention provide methods, apparatus, systems, computing devices, computing entities, and/or the like for generating a model deficiency data object for a tensor-based graph processing machine learning model. Certain embodiments of the present invention utilize systems, methods, and computer program products that generate a model deficiency data object for a tensor-based graph processing machine learning model using holistic graph links generated by utilizing a graph representation machine learning model.

### BACKGROUND

Various embodiments of the present invention address technical challenges related to performing predictive data analysis and provide solutions to address the efficiency and reliability shortcomings of existing predictive data analysis solutions.

### BRIEF SUMMARY

In general, various embodiments of the present invention provide methods, apparatus, systems, computing devices, computing entities, and/or the like for generating a model deficiency data object for a tensor-based graph processing machine learning model. Certain embodiments of the present invention utilize systems, methods, and computer program products that generate a model deficiency data object for a tensor-based graph processing machine learning model using holistic graph links generated by utilizing a graph representation machine learning model.

In accordance with one aspect, a method is provided. In one embodiment, the method comprises: identifying a positive input set that is associated with the risk category, wherein the positive input set comprises a plurality of prediction input data objects that are associated with an affirmative label for the risk category, and each prediction input data object in the positive input set is associated with: (i) a prediction input feature set, (ii) a plurality of risk tensors each generated based at least in part on a categorical subset of the prediction input feature set for the prediction input data object that is associated with an input category of a plurality of input categories, and (iii) a plurality of tensor-based graph representations generated based at least in part on the plurality of risk tensors for the prediction input data object; identifying a tensor-based graph representation set for the positive input set, wherein: (i) the tensor-based graph representation set comprises, for each prediction input data object in the positive input set, the plurality of tensor-based graph representations for the prediction input data object, and (ii) the tensor-based graph representation set describes a group of tensor-based graph links; generating, using a graph representation machine learning model, and based at least in part on each prediction input feature set, a group of holistic graph links; generating, based at least in part on the group of tensor-based graph links and the group of holistic graph links, a model deficiency data object; and performing one or more prediction-based actions based at least in part on the model deficiency data object.

In accordance with another aspect, a computer program product is provided. The computer program product may comprise at least one computer-readable storage medium having computer-readable program code portions stored therein, the computer-readable program code portions comprising executable portions configured to: identify a positive input set that is associated with the risk category, wherein: the positive input set comprises a plurality of prediction input data objects that are associated with an affirmative label for the risk category, and each prediction input data object in the positive input set is associated with: (i) a prediction input feature set, (ii) a plurality of risk tensors each generated based at least in part on a categorical subset of the prediction input feature set for the prediction input data object that is associated with an input category of a plurality of input categories, and (iii) a plurality of tensor-based graph representations generated based at least in part on the plurality of risk tensors for the prediction input data object; identify a tensor-based graph representation set for the positive input set, wherein: (i) the tensor-based graph representation set comprises, for each prediction input data object in the positive input set, the plurality of tensor-based graph representations for the prediction input data object, and (ii) the tensor-based graph representation set describes a group of tensor-based graph links; generate, using a graph representation machine learning model, and based at least in part on each prediction input feature set, a group of holistic graph links; generate, based at least in part on the group of tensor-based graph links and the group of holistic graph links, a model deficiency data object; and perform one or more prediction-based actions based at least in part on the model deficiency data object.

In accordance with yet another aspect, an apparatus comprising at least one processor and at least one memory including computer program code is provided. In one embodiment, the at least one memory and the computer program code may be configured to, with the processor, cause the apparatus to: identify a positive input set that is associated with the risk category, wherein: the positive input set comprises a plurality of prediction input data objects that are associated with an affirmative label for the risk category, and each prediction input data object in the positive input set is associated with: (i) a prediction input feature set, (ii) a plurality of risk tensors each generated based at least in part on a categorical subset of the prediction input feature set for the prediction input data object that is associated with an input category of a plurality of input categories, and (iii) a plurality of tensor-based graph representations generated based at least in part on the plurality of risk tensors for the prediction input data object; identify a tensor-based graph representation set for the positive input set, wherein: (i) the tensor-based graph representation set comprises, for each prediction input data object in the positive input set, the plurality of tensor-based graph representations for the prediction input data object, and (ii) the tensor-based graph representation set describes a group of tensor-based graph links; generate, using a graph representation machine learning model, and based at least in part on each prediction input feature set, a group of holistic graph links; generate, based at least in part on the group of tensor-based graph links and the group of holistic graph links, a model deficiency data object; and perform one or more prediction-based actions based at least in part on the model deficiency data object.

### BRIEF DESCRIPTION OF THE DRAWINGS

Having thus described the invention in general terms, reference will now be made to the accompanying drawings, which are not necessarily drawn to scale, and wherein:

FIG. 1 provides an exemplary overview of an architecture that can be used to practice embodiments of the present invention.

FIG. 2 provides an example predictive data analysis computing entity in accordance with some embodiments discussed herein.

FIG. 3 provides an example client computing entity in accordance with some embodiments discussed herein.

FIG. 4 is a flowchart diagram of an example process for generating a model deficiency data object for a tensor-based graph processing machine learning model that is associated with a risk category in accordance with some embodiments discussed herein.

FIG. 5 is a flowchart diagram of an example process for generating a group of holistic graph links for a risk category in accordance with some embodiments discussed herein.

FIG. 6 is a flowchart diagram of an example process for generating a model deficiency data object for a risk category based at least in part on a group of holistic graph links for the risk category in accordance with some embodiments discussed herein.

FIG. 7 is a flowchart diagram of an example process for generating a hybrid risk score generation machine learning model in accordance with some embodiments discussed herein.

FIG. 8 is a flowchart diagram of an example process for generating a tensor-based graph processing machine learning model in accordance with some embodiments discussed herein.

FIG. 9 is a flowchart diagram of an example process for generating a hybrid risk score generation machine learning model based at least in part on inferred hybrid risk scores for a set of prior patient data objects in accordance with some embodiments discussed herein.

FIG. 10 is a flowchart diagram of an example process for performing a model generation epoch of a genetic programming routine in accordance with some embodiments discussed herein.

FIG. 11 provides an operational example of a prediction output user interface in accordance with some embodiments discussed herein.

### DETAILED DESCRIPTION

Various embodiments of the present invention now will be described more fully hereinafter with reference to the accompanying drawings, in which some, but not all, embodiments of the invention are shown. Indeed, the invention may be embodied in many different forms and should not be construed as limited to the embodiments set forth herein; rather, these embodiments are provided so that this disclosure will satisfy applicable legal requirements. The term “or” is used herein in both the alternative and conjunctive sense, unless otherwise indicated. The terms “illustrative” and “exemplary” are used to be examples with no indication of quality level. Like numbers refer to like elements throughout. Moreover, while certain embodiments of the present invention are described with reference to predictive data analysis, one of ordinary skill in the art will recognize that the disclosed concepts can be used to perform other types of data analysis.

### I. Overview and Technical Improvements

Various embodiments of the present invention introduce techniques that improve the training speed of a graph processing machine learning framework given a constant/target predictive accuracy by using a model deficiency data object that is generated for the graph processing machine learning framework using holistic graph links inferred by a graph representation machine learning model. The combination of the noted components enables the proposed graph processing machine learning framework to generate more accurate graph-based predictions, which in turn increases the training speed of the proposed graph processing machine learning framework given a constant predictive accuracy. It is well-understood in the relevant art that there is typically a tradeoff between predictive accuracy and training speed, such that it is trivial to improve training speed by reducing predictive accuracy, and thus the real challenge is to improve training speed without sacrificing predictive accuracy through innovative model architectures. See, e.g., Sun et al., Feature-Frequency—Adaptive On-line Training for Fast and Accurate Natural Language Processing in 40(3) Computational Linguistic 563 at Abst. (“Typically, we need to make a tradeoff between speed and accuracy. It is trivial to improve the training speed via sacrificing accuracy or to improve the accuracy via sacrificing speed. Nevertheless, it is nontrivial to improve the training speed and the accuracy at the same time”). Accordingly, techniques that improve predictive accuracy without harming training speed, such as various techniques described herein, enable improving training speed given a constant predictive accuracy. Therefore, by improving accuracy of performing graph-based machine learning predictions, various embodiments of the present invention improve the training speed of graph processing machine learning frameworks given a constant/target predictive accuracy.

Various embodiments of the present invention make substantial technical improvements to performing operational load balancing for the post-prediction systems that perform post-prediction operations (e.g., automated specialist appointment scheduling operations) based at least in part on graph-based predictions. For example, in some embodiments, a predictive recommendation computing entity determines D classifications for D prediction input data objects using a graph processing machine learning framework that is augmented by a model deficiency data object that is generated for the graph processing machine learning framework using holistic graph links inferred by a graph representation machine learning model. Then, the count of D prediction input data objects that are associated with an affirmative classification, along with a resource utilization ratio for each prediction input data object, can be used to predict a predicted number of computing entities needed to perform post-prediction processing operations with respect to the D prediction input data objects. For example, in some embodiments, the number of computing entities needed to perform post-prediction processing operations (e.g., automated specialist scheduling operations) with respect to D prediction input data objects can be determined based at least in part on the output of the equation: R=ceil(EΣₖ^{k=K} urₖ), where R is the predicted number of computing entities needed to perform post-prediction processing operations with respect to the D prediction input data objects, cello) is a ceiling function that returns the closest integer that is greater than or equal to the value provided as the input parameter of the ceiling function, k is an index variable that iterates over K prediction input data objects among the D prediction input data objects that are associated with affirmative classifications, and urₖ is the estimated resource utilization ratio for a kth prediction input data object that may be determined based at least in part on a patient history complexity of a patient associated with the prediction input data object. In some embodiments, once R is generated, a predictive recommendation computing entity can use R to perform operational load balancing for a server system that is configured to perform post-prediction processing operations with respect to D prediction input data objects. This may be done by allocating computing entities to the post-prediction processing operations if the number of currently-allocated computing entities is below R, and deallocating currently-allocated computing entities if the number of currently-allocated computing entities is above R.

### II. Definitions of Certain Terms

The term “prediction input data object” may refer to a data entity that describes a real-world entity and/or a virtual entity with respect to which one or more predictive data analysis operations are performed in order to generate one or more predictive outputs (e.g., a hybrid risk score) for the prediction input data object. An example of a prediction input data object is a patient data object that describes one or more features associated with a particular patient/individual. In some embodiments, features associated with a patient data object include at least one of genomic data associated with the patient data object, behavioral data associated with the patient data object, clinical data associated with the patient data object, demographic data associated with the patient data object, health history data associated with the patient data object, and/or the like. In some embodiments, feature data described by a prediction input data object are referred to herein as the prediction input feature set for the prediction input data object. In some embodiments, each feature described by a prediction input feature set for a prediction input data object belong to an input category that describes a category of heterogenous feature data described by prediction input data objects.

The term “risk category” may refer to a data entity that describes a label space comprising a set of candidate labels, where the predictive outputs associated with a prediction input data object may be used to assign one candidate label in the label space to the prediction input data object. For example, a particular risk category may be associated with a particular disease/condition and describe a label space comprising a first candidate label that is assigned to a prediction input data object if the prediction input data object is predicted, based at least in part on the predictive outputs (e.g., the hybrid risk score) for the prediction input data object, to be at a high risk of the particular disease/condition and a second candidate label that is assigned to a prediction input data object if the prediction input data object is predicted, based at least in part on the predictive outputs (e.g., the hybrid risk score) for the prediction input data object, to be at a low risk of the particular disease/condition. As another example, a particular risk category may be associated with a particular disease/condition and describe a label space comprising a first candidate label that is assigned to a prediction input data object if the prediction input data object is predicted, based at least in part on the predictive outputs (e.g., the hybrid risk score) for the prediction input data object, to be at a high risk of the particular disease/condition, a second candidate label that is assigned to a prediction input data object if the prediction input data object is predicted, based at least in part on the predictive outputs (e.g., the hybrid risk score) for the prediction input data object, to be at a low risk of the particular disease/condition, and a third candidate label that is assigned to a prediction input data object if the prediction input data object is predicted, based at least in part on the predictive outputs (e.g., the hybrid risk score) for the prediction input data object, to be at a medium risk of the particular disease/condition.

The term “positive input set” may refer to a data entity that describes a set of prediction input data objects (e.g., a set of patient input data objects) that are associated with an affirmative label defined by a corresponding risk category. For example, the positive input set for a risk category that is associated with a particular disease/condition may include a set of patient data objects associated with a set of patients/individuals that suffer from the particular disease/condition. As another example, the positive input set for a risk category that is associated with a particular disease/condition may include a set of patient data objects associated with a set of patients/individuals that are predicted to be associated with a high risk of developing the particular disease/condition. As described above, in some embodiments, a risk category is associated with a label space defining a set of candidate labels. In some of the noted embodiments, for a given risk category, one of the candidate labels defined by the label space for the given risk category is designated as an affirmative label, and then a set of prediction input data objects whose ground-truth labels correspond to the affirmative label are designated as the prediction input data objects in the positive input sets for the given risk category. For example, when a risk category is associated with a label space for a particular disease/condition that comprises a high-risk label and a low-risk label, the high-risk label may be designated as an affirmative label, and thus those prediction input data objects whose ground-truth labels correspond to the high-risk label may be designated as the positive input set for the noted risk category.

The term “holistic graph link” may refer to a data entity that describes a relationship between two or more features described by the prediction input feature sets for the prediction input data objects in the positive input set for a risk category, where the relationship is generated based at least in part on filtering pairwise relationships between features described by the prediction input feature sets in accordance with predicted relevance measures for the noted pairwise relationships. For example, co-occurrence edge weights generated by a graph representation machine learning model based at least in part on prediction input feature sets for the high-risk population associated with a particular disease/condition may describe that: (i) there is a sufficiently strong correlation between detection of a particular genetic variant in the genomic sequences of the high-risk population and detection of high smoking behavior in the high-risk population, (ii) there is a sufficiently strong correlation between detection of high amounts of usage of a particular inhaler in the high-risk population and detection of the particular genetic variant in the genomic sequences of the high-risk population, and (iii) there is a sufficiently strong correlation between detection of high amounts of usage of the particular inhaler in the high-risk population and detection of high smoking behavior in the high-risk population. In some embodiments, given the noted predictive inferences, a holistic graph link may be generated for the feature set comprising a first feature corresponding to detection of the particular genetic variant, a second feature corresponding to the detection of high smoking behavior, and a third feature corresponding to detection of high amounts of usage of the particular inhaler.

The term “graph representation machine learning model” may refer to a data entity that describes a machine learning model, where the machine learning model is configured to generate a co-occurrence edge weight for a co-occurrence edge between two node features. In some embodiments, given F features and P prediction input feature sets that are associated with the positive input set for a risk category, to generate the co-occurrence edge weight for a co-occurrence edge between a first feature node associated with a first feature and a second feature node associated with a second feature, a graph representation machine learning model first generates P per-input co-occurrence likelihood values for the feature pair comprising the first feature and the second feature, with each per-input co-occurrence likelihood value being associated with a corresponding prediction input feature set in P prediction input feature sets that are associated with the positive input set and describing a predicted likelihood that the prediction input feature set describes occurrence/detection of both the second feature and the second feature. Then, to generate the co-occurrence edge weight for the noted co-occurrence edge, the graph representation machine learning model: (i) aggregates all of the P per-input co-occurrence likelihood values for the feature pair comprising the first feature and the second feature to generate a cross-input co-occurrence likelihood value, and (ii) normalizes the

$$\frac{F !}{2 ( F - 2 ) !}$$

cross-input co-occurrence likelihood values across the

$$\frac{F !}{2 ( F - 2 ) !}$$

feature pairs to generate

$$\frac{F !}{2 ( F - 2 ) !}$$

co-occurrence edge weights for the

$$\frac{F !}{2 ( F - 2 ) !}$$

co-occurrence edges. In some embodiments, inputs to the graph representation machine learning model comprise T vectors, where T may define the number of input tokens generated based at least in part on input prediction input feature sets or the number of input tokens generated based at least in part on all of the prediction input feature sets depending on the embodiments. In some embodiments, outputs of the graph representation machine learning model comprise a vector describing

$$\frac{F !}{2 ( F - 2 ) !}$$

per-input co-occurrence likelihood values for an input prediction input feature set across

$$\frac{F !}{2 ( F - 2 ) !}$$

distinctive feature pairs defined for F features in a relevant feature space. In some embodiments, the graph representation machine learning model is defined using training data describing ground-truth observations about co-occurrence of features in particular real-world entities and/or particular virtual-world entities (e.g., ground-truth observations about co-occurrence of features across input categories in a particular patient data object).

The term “input category” may refer to a data entity that describes a defined attribute of a feature in a prediction input feature set for a prediction input data object that is used to divide the prediction input feature set into categorical subsets that are in turn used to generate risk tensors. Examples of input categories for the prediction input feature set for a patient input data object include at least one of a genomic data input category, a behavioral data risk category, a clinical data risk category, a demographic data risk category, a health history data risk category, and/or the like. In some embodiments, given a positive input set for a risk category that comprises P prediction input data objects (e.g., corresponding to P patients/individuals that suffer from a particular disease/condition), P prediction input feature sets are identified, where each prediction input feature set comprises the prediction input features for a corresponding prediction input data object. Importantly, in some embodiments, the prediction input features for a particular prediction input data object comprise features associated with C input categories. In some of the noted embodiments, holistic graph links are generated based at least in part on co-occurrence edge weights that are generated based at least in part on the totality of each of the P prediction input feature sets, which includes each feature described by the P prediction input feature sets regardless of input category. In other words, if S_{a,b} describes features of an ath prediction input data object that belong to a bth input category, co-occurrence edge weights that are used to generate holistic graph links are generated based at least in part on a feature set that comprises Uᵢ₌₁^{P}Uⱼ₌₁^{C}S_{i,j}. This is an important property, because it means that holistic graph links are able to capture predictive associations across prediction input data objects as well as predictive associations across input categories, which means that the holistic graph links can include predictive insights that are not captured by tensor-based graph representations used to generate input data for a particular prediction input data that is provided to a tensor-based graph processing machine learning model.

The term “tensor-based graph link” may refer to a data entity that describes a feature set whose collective relationship is captured by at least one tensor-based graph representation in the tensor-based graph representation set for a corresponding risk category. In some embodiments, because the at least one tensor-based graph representation in the tensor-based graph representation set for a corresponding risk category captures the collective relationship associated with the feature set, the predictive data analysis computing entity 106 infers that the collective relationship is captured by the risk tensors used to generate input data for a tensor-based graph processing machine learning model that is associated with the corresponding risk category, and thus the tensor-based graph processing machine learning model that is associated with the corresponding risk category is not deficient with respect to the noted collective relationship. In some embodiments, a tensor-based graph link describes a maximally-sized fully-connected subgraph of a tensor-based graph representation in the tensor-based graph representation set for a corresponding risk category. For example, consider an exemplary embodiment in which the set of tensor-based graph edges described by the tensor-based graph representations in the tensor-based graph representation set for a corresponding risk category include O_{1,2}, O_{1,3}, O_{2,3}, and O_{3,4}. In this example, the tensor-based graph representation set is associated with a first tensor-based graph link that is associated with the features F₁, F₂, and F₃, and a second tensor-based graph link that is associated with the features F₃ and F₄. In some embodiments, a tensor-based graph link describes a fully-connected subgraph of a tensor-based graph representation in the tensor-based graph representation set for a corresponding risk category. For example, consider an exemplary embodiment in which the set of tensor-based graph edges described by the tensor-based graph representations in in the tensor-based graph representation set for a corresponding risk category include O_{1,2}, O_{1,3}, O_{2,3}, and O_{3,4}. In this example, the tensor-based graph representation set is associated with a first tensor-based graph link that is associated with the features F₁, F₂, and F₃, a second tensor-based graph link that is associated with the features F₃ and F₄, a third tensor-based graph link that is associated with the features F₁ and F₂, a fourth tensor-based graph link that is associated with the features F₁ and F₃, and a fifth tensor-based graph link that is associated with the features F₂ and F₃.

The term “prevalence score” may refer to a data entity that describes an estimated measure of the ratio of a predictive input population (e.g., a patient/individual population) that has features corresponding to the feature set for a deficiency graph link to a total predictive input population. In some embodiments, the prevalence score for a deficiency graph link describes whether the feature set associated with the deficiency graph link is present in a statistically significant portion of the population. In some embodiments, by factoring in the rarity of the disease (e.g., as calculated via analysis of clinical research) associated with the risk category, the predictive data analysis computing entity 106 determines if the risk factors associated with the feature set for a deficiency graph link are present in a statistically significant portion of the population. This is a critical step as selecting a smaller population size may be feasible for a rare disease, but a more common disease would need a much larger population size to ensure that it was representative of the general population with the disease in question. If multiple risk factors are found to be present in a statistically significant portion of the population, then those risk factors could be studied as a group rather than individually. This would aid in the understanding of complex diseases that have multiple genetic variants that influence their severity and treatment, such as the dystrophy gene. In some embodiments, if an identified risk factor is suspected to be of significance by either manual research or the steps described above but falls short of statistical significance for the population, that risk factor can be revisited at a later date to ensure that the further analysis would be meaningful.

The term “tensor-based graph processing machine learning model” may refer to a data entity that describes parameters, hyper-parameters, and/or defined operations of a machine learning model, where the machine learning model is configured to process one or more tensor-based graph feature embeddings for a prediction input data object in order to generate an inferred hybrid risk score for the prediction input data object. A trained tensor-based graph processing machine learning model may be configured to receive, as at least a part of its inputs, one or more tensor-based graph feature embeddings for a prediction input data object, where a tensor-based graph feature embedding may be a vector of one or more values that are determined based at least in part on a tensor-based graph representation of a risk tensor of one or more risk tensors for the prediction input data object. In some embodiments, the trained tensor-based graph processing machine learning model may include: a plurality of graph-based machine learning models (e.g., including one or more graph convolutional neural network machine learning models), where each graph-based machine learning model is configured to process a tensor-based graph feature embedding for a particular input category to generate a per-model machine learning output, and an ensemble machine learning model that is configured to aggregate/combine per-model machine learning outputs across various graph-based machine learning models to generate the inferred hybrid risk score for the prediction input data object. For example, a trained tensor-based graph processing machine learning model may include: a first graph-based machine learning model that is configured to process a tensor-based graph feature embedding determined based at least in part on genomic data (e.g., based at least in part on a genomic risk tensor) for a prediction input data object in order to generate a first per-model machine learning output, a second graph-based machine learning model that is configured to process a tensor-based graph feature embedding determined based at least in part on clinical data (e.g., based at least in part on a clinical risk tensor) for a prediction input data object in order to generate a second per-model machine learning output, a third graph-based machine learning model that is configured to process a tensor-based graph feature embedding determined based at least in part on behavioral data (e.g., based at least in part on a behavioral risk tensor) for a prediction input data object in order to generate a third per-model machine learning output, and an ensemble machine learning model that is configured to aggregate/combine the first per-model machine learning output, the second per-model machine learning output, and the third per-model machine learning output in order to generate the inferred hybrid risk score for the prediction input data object. In some embodiments, the trained tensor-based graph processing machine learning model is trained (e.g., via one or more end-to-end training operations) using ground-truth hybrid risk scores for a set of ground-truth tensor-based graph feature embeddings for each training prediction input data object of a set of training prediction input data objects.

The term “risk tensor” may refer to a data entity that describes a tensor data object that includes a set of subject-matter-defined data items associated with a prediction input data object. In some embodiments, a risk tensor describes a heterogeneous group of data that are related by the underlying risk tensor and relate to an input category that corresponds to the risk tensor. Examples of risk tensors include a genomic risk tensor that includes genomic data associated with a prediction input data object, a behavioral risk tensor that includes behavioral data associated with a prediction input data object, a clinical risk tensor that includes clinical data associated with a prediction input data object, a demographic risk tensor that includes demographic data associated with a prediction input data object, a health history risk tensor that includes health history data associated with a prediction input data object, and/or the like.

The term “tensor-based graph feature embedding” may refer to a data entity that describes a fixed-size representation of a tensor-based graph representation, which is a graph-based representation of a risk tensor. In some embodiments, to generate a graph-based representation of a risk tensor, the predictive data analysis computing entity 106 may embed/convert/transform data items in the given risk tensor into a graph (e.g., a multiplex graph) representation (e.g., by converting the risk tensor into a graph embedding, for example by using a Node2Vec feature embedding routine). For example, given a genomic risk tensor, if suitable genomic networks for any diseases under consideration are available from the Kyoto Encyclopedia of Genes and Genomes (KEGG) resource (e.g. for non-small cell lung cancer, the genomic pathway, available online at https://www.genome.jp/kegg-bin/show_pathway?hsa05223), then the pathway may be converted to a graph representation in order to generate a tensor-based graph feature embedding for the genomic risk tensor. In some embodiments, when a tensor-based graph feature embedding is used to generate a trained tensor-based graph processing machine learning model (e.g., by using ground-truth tensor-based graph feature embeddings as inputs during the training of a tensor-based graph processing machine learning model), the tensor-based graph feature embedding is referred to as a ground-truth tensor-based graph feature embedding. In some embodiments, when a tensor-based graph feature embedding is used to generate inferred hybrid risk scores that are in turn used to generate a hybrid risk score generation machine learning model, the tensor-based graph feature embedding is referred to as a prior tensor-based graph feature embedding.

The term “hybrid risk score generation machine learning model” may refer to a data entity that describes parameters, hyper-parameters, and/or defined operations of a machine learning model, where the machine learning model is configured to relate one or more tensor-based graph feature embeddings for a prediction input data object to a hybrid risk score for the prediction input data object. For example, the hybrid risk score generation machine learning model may be determined by performing one or more genetic programming operations (e.g., including one or more symbolic regression operations) based at least in part on sets of prior tensor-based graph feature embeddings for a set of prior prediction input data objects and a set of corresponding inferred hybrid risk scores for the set of prior prediction input data objects, where an inferred hybrid risk score for a prior prediction input data object may be determined by processing the set of prior tensor-based graph feature embeddings for the prior prediction input data object using a trained tensor-based graph processing machine learning model, and where the set of prior tensor-based graph feature embeddings for a prior prediction input data object may be supplied as input variables and/or as regressor variables for the one or more genetic programming operations performed to generate the hybrid risk score generation machine learning model. The hybrid risk score generation machine learning model may be configured to process, as inputs, a set of tensor-based graph feature embeddings and generate, as an output, a hybrid risk score, where each tensor-based graph feature embedding may be a vector, and where each hybrid risk score may be an atomic value or a vector.

The term “inferred hybrid risk score” may refer to a data entity that describes a risk score that is generated by a trained tensor-based graph processing machine learning model by processing a set of tensor-based graph feature embeddings for a corresponding prediction input data object. For example, the inferred hybrid risk score for a particular prediction input data object may be generated by processing (using a trained tensor-based graph processing machine learning model) the genomic tensor-based graph feature embedding for the particular prediction input data object as determined based at least in part on the genomic risk tensor for the particular prediction input data object, the clinical tensor-based graph feature embedding for the particular prediction input data object as determined based at least in part on the clinical risk tensor for the particular prediction input data object, and the behavioral tensor-based graph feature embedding for the particular prediction input data object based at least in part on the behavioral risk tensor for the particular prediction input data object. The inferred hybrid risk score may be a vector.

### III. Computer Program Products, Methods, and Computing Entities

Embodiments of the present invention may be implemented in various ways, including as computer program products that comprise articles of manufacture. Such computer program products may include one or more software components including, for example, software objects, methods, data structures, or the like. A software component may be coded in any of a variety of programming languages. An illustrative programming language may be a lower-level programming language such as an assembly language associated with a particular hardware architecture and/or operating system platform. A software component comprising assembly language instructions may require conversion into executable machine code by an assembler prior to execution by the hardware architecture and/or platform. Another example programming language may be a higher-level programming language that may be portable across multiple architectures. A software component comprising higher-level programming language instructions may require conversion to an intermediate representation by an interpreter or a compiler prior to execution.

Other examples of programming languages include, but are not limited to, a macro language, a shell or command language, a job control language, a script language, a database query or search language, and/or a report writing language. In one or more example embodiments, a software component comprising instructions in one of the foregoing examples of programming languages may be executed directly by an operating system or other software component without having to be first transformed into another form. A software component may be stored as a file or other data storage construct. Software components of a similar type or functionally related may be stored together such as, for example, in a particular directory, folder, or library. Software components may be static (e.g., pre-established or fixed) or dynamic (e.g., created or modified at the time of execution).

A computer program product may include a non-transitory computer-readable storage medium storing applications, programs, program modules, scripts, source code, program code, object code, byte code, compiled code, interpreted code, machine code, executable instructions, and/or the like (also referred to herein as executable instructions, instructions for execution, computer program products, program code, and/or similar terms used herein interchangeably). Such non-transitory computer-readable storage media include all computer-readable media (including volatile and non-volatile media).

In one embodiment, a non-volatile computer-readable storage medium may include a floppy disk, flexible disk, hard disk, solid-state storage (SSS) (e.g., a solid state drive (SSD), solid state card (SSC), solid state module (SSM), enterprise flash drive, magnetic tape, or any other non-transitory magnetic medium, and/or the like. A non-volatile computer-readable storage medium may also include a punch card, paper tape, optical mark sheet (or any other physical medium with patterns of holes or other optically recognizable indicia), compact disc read only memory (CD-ROM), compact disc-rewritable (CD-RW), digital versatile disc (DVD), Blu-ray disc (BD), any other non-transitory optical medium, and/or the like. Such a non-volatile computer-readable storage medium may also include read-only memory (ROM), programmable read-only memory (PROM), erasable programmable read-only memory (EPROM), electrically erasable programmable read-only memory (EEPROM), flash memory (e.g., Serial, NAND, NOR, and/or the like), multimedia memory cards (MMC), secure digital (SD) memory cards, SmartMedia cards, CompactFlash (CF) cards, Memory Sticks, and/or the like. Further, a non-volatile computer-readable storage medium may also include conductive-bridging random access memory (CBRAM), phase-change random access memory (PRAM), ferroelectric random-access memory (FeRAM), non-volatile random-access memory (NVRAM), magnetoresistive random-access memory (MRAM), resistive random-access memory (RRAM), Silicon-Oxide-Nitride-Oxide-Silicon memory (SONOS), floating junction gate random access memory (FJG RAM), Millipede memory, racetrack memory, and/or the like.

In one embodiment, a volatile computer-readable storage medium may include random access memory (RAM), dynamic random access memory (DRAM), static random access memory (SRAM), fast page mode dynamic random access memory (FPM DRAM), extended data-out dynamic random access memory (EDO DRAM), synchronous dynamic random access memory (SDRAM), double data rate synchronous dynamic random access memory (DDR SDRAM), double data rate type two synchronous dynamic random access memory (DDR2 SDRAM), double data rate type three synchronous dynamic random access memory (DDR3 SDRAM), Rambus dynamic random access memory (RDRAM), Twin Transistor RAM (TTRAM), Thyristor RAM (T-RAM), Zero-capacitor (Z-RAM), Rambus in-line memory module (RIMM), dual in-line memory module (DIMM), single in-line memory module (SIMM), video random access memory (VRAM), cache memory (including various levels), flash memory, register memory, and/or the like. It will be appreciated that where embodiments are described to use a computer-readable storage medium, other types of computer-readable storage media may be substituted for or used in addition to the computer-readable storage media described above.

As should be appreciated, various embodiments of the present invention may also be implemented as methods, apparatus, systems, computing devices, computing entities, and/or the like. As such, embodiments of the present invention may take the form of an apparatus, system, computing device, computing entity, and/or the like executing instructions stored on a computer-readable storage medium to perform certain steps or operations. Thus, embodiments of the present invention may also take the form of an entirely hardware embodiment, an entirely computer program product embodiment, and/or an embodiment that comprises combination of computer program products and hardware performing certain steps or operations.

Embodiments of the present invention are described below with reference to block diagrams and flowchart illustrations. Thus, it should be understood that each block of the block diagrams and flowchart illustrations may be implemented in the form of a computer program product, an entirely hardware embodiment, a combination of hardware and computer program products, and/or apparatus, systems, computing devices, computing entities, and/or the like carrying out instructions, operations, steps, and similar words used interchangeably (e.g., the executable instructions, instructions for execution, program code, and/or the like) on a computer-readable storage medium for execution. For example, retrieval, loading, and execution of code may be performed sequentially such that one instruction is retrieved, loaded, and executed at a time. In some exemplary embodiments, retrieval, loading, and/or execution may be performed in parallel such that multiple instructions are retrieved, loaded, and/or executed together. Thus, such embodiments can produce specifically-configured machines performing the steps or operations specified in the block diagrams and flowchart illustrations. Accordingly, the block diagrams and flowchart illustrations support various combinations of embodiments for performing the specified instructions, operations, or steps.

### IV. Exemplary System Architecture

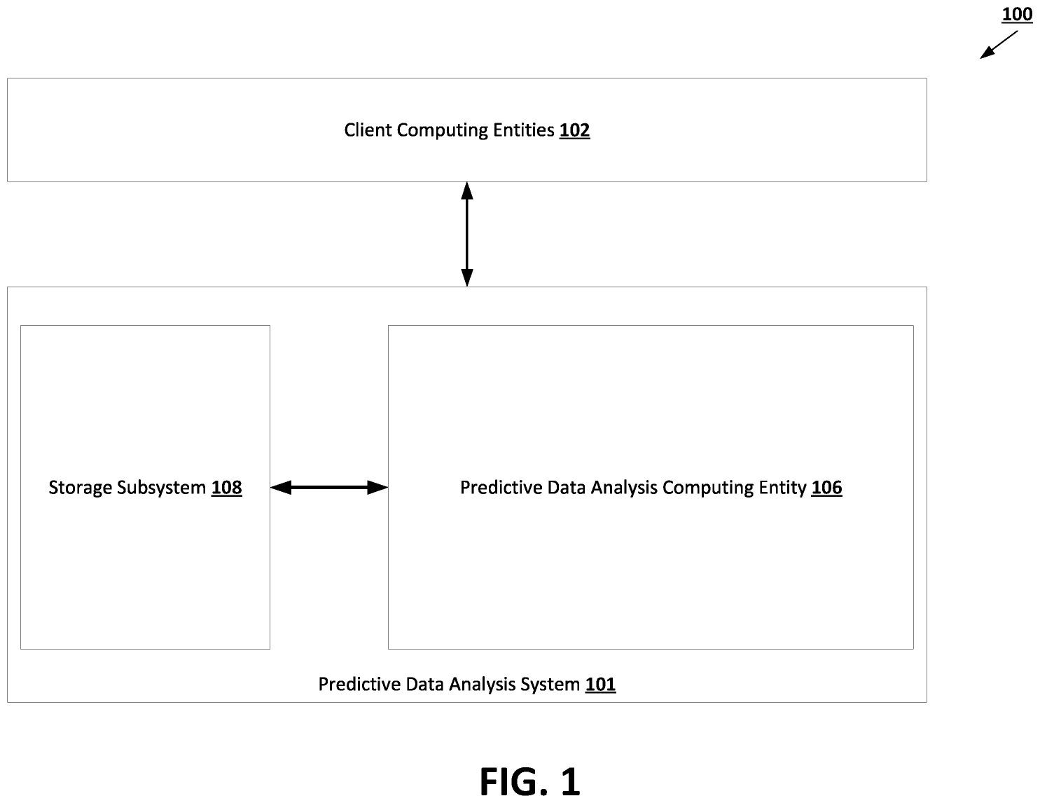

FIG. 1 is a schematic diagram of an example architecture 100 for performing predictive data analysis. The architecture 100 includes a predictive data analysis system 101 configured to receive predictive data analysis requests from client computing entities 102, process the predictive data analysis requests to generate predictions, provide the generated predictions to the client computing entities 102, and automatically perform prediction-based actions based at least in part on the generated predictions. An example of a prediction-based action that can be performed using the predictive data analysis system 101 is a request for generating a disease risk score based at least in part on at least one of patient genomic data, patient behavioral data, patient clinical data, and/or the like.

In some embodiments, predictive data analysis system 101 may communicate with at least one of the client computing entities 102 using one or more communication networks. Examples of communication networks include any wired or wireless communication network including, for example, a wired or wireless local area network (LAN), personal area network (PAN), metropolitan area network (MAN), wide area network (WAN), or the like, as well as any hardware, software and/or firmware required to implement it (such as, e.g., network routers, and/or the like).

The predictive data analysis system 101 may include a predictive data analysis computing entity 106 and a storage subsystem 108. The predictive data analysis computing entity 106 may be configured to receive predictive data analysis requests from one or more client computing entities 102, process the predictive data analysis requests to generate predictions corresponding to the predictive data analysis requests, provide the generated predictions to the client computing entities 102, and automatically perform prediction-based actions based at least in part on the generated predictions.

The storage subsystem 108 may be configured to store input data used by the predictive data analysis computing entity 106 to perform predictive data analysis as well as model definition data used by the predictive data analysis computing entity 106 to perform various predictive data analysis tasks. The storage subsystem 108 may include one or more storage units, such as multiple distributed storage units that are connected through a computer network. Each storage unit in the storage subsystem 108 may store at least one of one or more data assets and/or one or more data about the computed properties of one or more data assets. Moreover, each storage unit in the storage subsystem 108 may include one or more non-volatile storage or memory media including, but not limited to, hard disks, ROM, PROM, EPROM, EEPROM, flash memory, MMCs, SD memory cards, Memory Sticks, CBRAM, PRAM, FeRAM, NVRAM, MRAM, RRAM, SONOS, FJG RAM, Millipede memory, racetrack memory, and/or the like.

### A. Exemplary Predictive Data Analysis Computing Entity

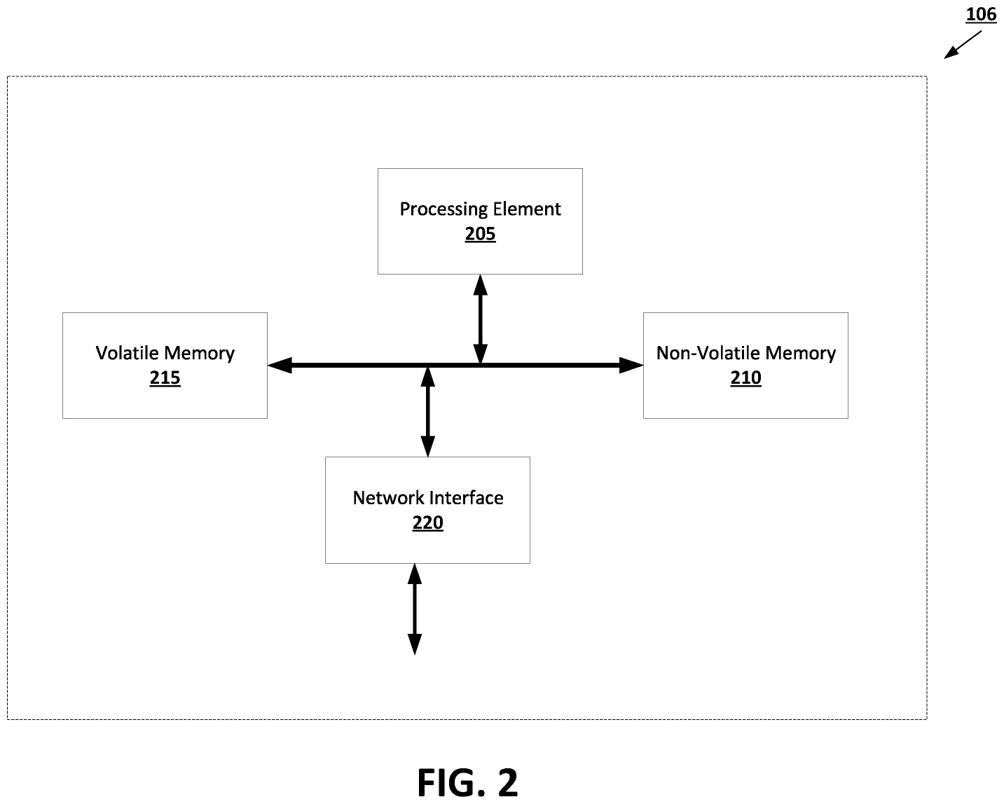

FIG. 2 provides a schematic of a predictive data analysis computing entity 106 according to one embodiment of the present invention. In general, the terms computing entity, computer, entity, device, system, and/or similar words used herein interchangeably may refer to, for example, one or more computers, computing entities, desktops, mobile phones, tablets, phablets, notebooks, laptops, distributed systems, kiosks, input terminals, servers or server networks, blades, gateways, switches, processing devices, processing entities, set-top boxes, relays, routers, network access points, base stations, the like, and/or any combination of devices or entities adapted to perform the functions, operations, and/or processes described herein. Such functions, operations, and/or processes may include, for example, transmitting, receiving, operating on, processing, displaying, storing, determining, creating/generating, monitoring, evaluating, comparing, and/or similar terms used herein interchangeably. In one embodiment, these functions, operations, and/or processes can be performed on data, content, information, and/or similar terms used herein interchangeably.

As indicated, in one embodiment, the predictive data analysis computing entity 106 may also include one or more communications interfaces 220 for communicating with various computing entities, such as by communicating data, content, information, and/or similar terms used herein interchangeably that can be transmitted, received, operated on, processed, displayed, stored, and/or the like.

As shown in FIG. 2, in one embodiment, the predictive data analysis computing entity 106 may include, or be in communication with, one or more processing elements 205 (also referred to as processors, processing circuitry, and/or similar terms used herein interchangeably) that communicate with other elements within the predictive data analysis computing entity 106 via a bus, for example. As will be understood, the processing element 205 may be embodied in a number of different ways.

For example, the processing element 205 may be embodied as one or more complex programmable logic devices (CPLDs), microprocessors, multi-core processors, coprocessing entities, application-specific instruction-set processors (ASIPs), microcontrollers, and/or controllers. Further, the processing element 205 may be embodied as one or more other processing devices or circuitry. The term circuitry may refer to an entirely hardware embodiment or a combination of hardware and computer program products. Thus, the processing element 205 may be embodied as integrated circuits, application specific integrated circuits (ASICs), field programmable gate arrays (FPGAs), programmable logic arrays (PLAs), hardware accelerators, other circuitry, and/or the like.

As will therefore be understood, the processing element 205 may be configured for a particular use or configured to execute instructions stored in volatile or non-volatile media or otherwise accessible to the processing element 205. As such, whether configured by hardware or computer program products, or by a combination thereof, the processing element 205 may be capable of performing steps or operations according to embodiments of the present invention when configured accordingly.

In one embodiment, the predictive data analysis computing entity 106 may further include, or be in communication with, non-volatile media (also referred to as non-volatile storage, memory, memory storage, memory circuitry and/or similar terms used herein interchangeably). In one embodiment, the non-volatile storage or memory may include one or more non-volatile storage or memory media 210, including, but not limited to, hard disks, ROM, PROM, EPROM, EEPROM, flash memory, MMCs, SD memory cards, Memory Sticks, CBRAM, PRAM, FeRAM, NVRAM, MRAM, RRAM, SONOS, FJG RAM, Millipede memory, racetrack memory, and/or the like.

As will be recognized, the non-volatile storage or memory media may store databases, database instances, database management systems, data, applications, programs, program modules, scripts, source code, object code, byte code, compiled code, interpreted code, machine code, executable instructions, and/or the like. The term database, database instance, database management system, and/or similar terms used herein interchangeably may refer to a collection of records or data that is stored in a computer-readable storage medium using one or more database models, such as a hierarchical database model, network model, relational model, entity—relationship model, object model, document model, semantic model, graph model, and/or the like.

In one embodiment, the predictive data analysis computing entity 106 may further include, or be in communication with, volatile media (also referred to as volatile storage, memory, memory storage, memory circuitry and/or similar terms used herein interchangeably). In one embodiment, the volatile storage or memory may also include one or more volatile storage or memory media 215, including, but not limited to, RAM, DRAM, SRAM, FPM DRAM, EDO DRAM, SDRAM, DDR SDRAM, DDR2 SDRAM, DDR3 SDRAM, RDRAM, TTRAM, T-RAM, Z-RAM, RIMM, DIMM, SIMM, VRAM, cache memory, register memory, and/or the like.

As will be recognized, the volatile storage or memory media may be used to store at least portions of the databases, database instances, database management systems, data, applications, programs, program modules, scripts, source code, object code, byte code, compiled code, interpreted code, machine code, executable instructions, and/or the like being executed by, for example, the processing element 205. Thus, the databases, database instances, database management systems, data, applications, programs, program modules, scripts, source code, object code, byte code, compiled code, interpreted code, machine code, executable instructions, and/or the like may be used to control certain aspects of the operation of the predictive data analysis computing entity 106 with the assistance of the processing element 205 and operating system.

As indicated, in one embodiment, the predictive data analysis computing entity 106 may also include one or more communications interfaces 220 for communicating with various computing entities, such as by communicating data, content, information, and/or similar terms used herein interchangeably that can be transmitted, received, operated on, processed, displayed, stored, and/or the like. Such communication may be executed using a wired data transmission protocol, such as fiber distributed data interface (FDDI), digital subscriber line (DSL), Ethernet, asynchronous transfer mode (ATM), frame relay, data over cable service interface specification (DOCSIS), or any other wired transmission protocol. Similarly, the predictive data analysis computing entity 106 may be configured to communicate via wireless external communication networks using any of a variety of protocols, such as general packet radio service (GPRS), Universal Mobile Telecommunications System (UMTS), Code Division Multiple Access 2000 (CDMA2000), CDMA2000 1× (1×RTT), Wideband Code Division Multiple Access (WCDMA), Global System for Mobile Communications (GSM), Enhanced Data rates for GSM Evolution (EDGE), Time Division-Synchronous Code Division Multiple Access (TD-SCDMA), Long Term Evolution (LTE), Evolved Universal Terrestrial Radio Access Network (E-UTRAN), Evolution-Data Optimized (EVDO), High Speed Packet Access (HSPA), High-Speed Downlink Packet Access (HSDPA), IEEE 802.11 (Wi-Fi), Wi-Fi Direct, 802.16 (WiMAX), ultra-wideband (UWB), infrared (IR) protocols, near field communication (NFC) protocols, Wibree, Bluetooth protocols, wireless universal serial bus (USB) protocols, and/or any other wireless protocol.

Although not shown, the predictive data analysis computing entity 106 may include, or be in communication with, one or more input elements, such as a keyboard input, a mouse input, a touch screen/display input, motion input, movement input, audio input, pointing device input, joystick input, keypad input, and/or the like. The predictive data analysis computing entity 106 may also include, or be in communication with, one or more output elements (not shown), such as audio output, video output, screen/display output, motion output, movement output, and/or the like.

### B. Exemplary Client Computing Entity

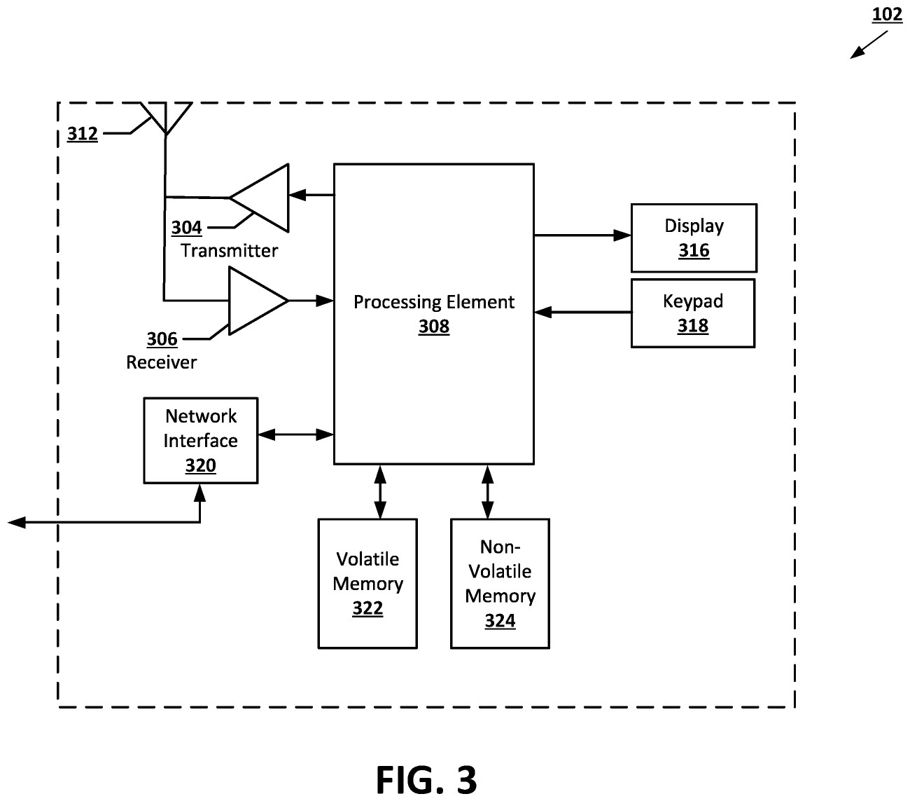

FIG. 3 provides an illustrative schematic representative of an client computing entity 102 that can be used in conjunction with embodiments of the present invention. In general, the terms device, system, computing entity, entity, and/or similar words used herein interchangeably may refer to, for example, one or more computers, computing entities, desktops, mobile phones, tablets, phablets, notebooks, laptops, distributed systems, kiosks, input terminals, servers or server networks, blades, gateways, switches, processing devices, processing entities, set-top boxes, relays, routers, network access points, base stations, the like, and/or any combination of devices or entities adapted to perform the functions, operations, and/or processes described herein. Client computing entities 102 can be operated by various parties. As shown in FIG. 3, the client computing entity 102 can include an antenna 312, a transmitter 304 (e.g., radio), a receiver 306 (e.g., radio), and a processing element 308 (e.g., CPLDs, microprocessors, multi-core processors, coprocessing entities, ASIPs, microcontrollers, and/or controllers) that provides signals to and receives signals from the transmitter 304 and receiver 306, correspondingly.

The signals provided to and received from the transmitter 304 and the receiver 306, correspondingly, may include signaling information/data in accordance with air interface standards of applicable wireless systems. In this regard, the client computing entity 102 may be capable of operating with one or more air interface standards, communication protocols, modulation types, and access types. More particularly, the client computing entity 102 may operate in accordance with any of a number of wireless communication standards and protocols, such as those described above with regard to the predictive data analysis computing entity 106. In a particular embodiment, the client computing entity 102 may operate in accordance with multiple wireless communication standards and protocols, such as UMTS, CDMA2000, 1×RTT, WCDMA, GSM, EDGE, TD-SCDMA, LTE, E-UTRAN, EVDO, HSPA, HSDPA, Wi-Fi, Wi-Fi Direct, WiMAX, UWB, IR, NFC, Bluetooth, USB, and/or the like. Similarly, the client computing entity 102 may operate in accordance with multiple wired communication standards and protocols, such as those described above with regard to the predictive data analysis computing entity 106 via a network interface 320.

Via these communication standards and protocols, the client computing entity 102 can communicate with various other entities using concepts such as Unstructured Supplementary Service Data (USSD), Short Message Service (SMS), Multimedia Messaging Service (MMS), Dual-Tone Multi-Frequency Signaling (DTMF), and/or Subscriber Identity Module Dialer (SIM dialer). The client computing entity 102 can also download changes, add-ons, and updates, for instance, to its firmware, software (e.g., including executable instructions, applications, program modules), and operating system.

According to one embodiment, the client computing entity 102 may include location determining aspects, devices, modules, functionalities, and/or similar words used herein interchangeably. For example, the client computing entity 102 may include outdoor positioning aspects, such as a location module adapted to acquire, for example, latitude, longitude, altitude, geocode, course, direction, heading, speed, universal time (UTC), date, and/or various other information/data. In one embodiment, the location module can acquire data, sometimes known as ephemeris data, by identifying the number of satellites in view and the relative positions of those satellites (e.g., using global positioning systems (GPS)). The satellites may be a variety of different satellites, including Low Earth Orbit (LEO) satellite systems, Department of Defense (DOD) satellite systems, the European Union Galileo positioning systems, the Chinese Compass navigation systems, Indian Regional Navigational satellite systems, and/or the like. This data can be collected using a variety of coordinate systems, such as the Decimal Degrees (DD); Degrees, Minutes, Seconds (DMS); Universal Transverse Mercator (UTM); Universal Polar Stereographic (UPS) coordinate systems; and/or the like. Alternatively, the location information/data can be determined by triangulating the client computing entity's 102 position in connection with a variety of other systems, including cellular towers, Wi-Fi access points, and/or the like. Similarly, the client computing entity 102 may include indoor positioning aspects, such as a location module adapted to acquire, for example, latitude, longitude, altitude, geocode, course, direction, heading, speed, time, date, and/or various other information/data. Some of the indoor systems may use various position or location technologies including RFID tags, indoor beacons or transmitters, Wi-Fi access points, cellular towers, nearby computing devices (e.g., smartphones, laptops) and/or the like. For instance, such technologies may include the iBeacons, Gimbal proximity beacons, Bluetooth Low Energy (BLE) transmitters, NFC transmitters, and/or the like. These indoor positioning aspects can be used in a variety of settings to determine the location of someone or something to within inches or centimeters.

The client computing entity 102 may also comprise a user interface (that can include a display 316 coupled to a processing element 308) and/or a user input interface (coupled to a processing element 308). For example, the user interface may be a user application, browser, user interface, and/or similar words used herein interchangeably executing on and/or accessible via the client computing entity 102 to interact with and/or cause display of information/data from the predictive data analysis computing entity 106, as described herein. The user input interface can comprise any of a number of devices or interfaces allowing the client computing entity 102 to receive data, such as a keypad 318 (hard or soft), a touch display, voice/speech or motion interfaces, or other input device. In embodiments including a keypad 318, the keypad 318 can include (or cause display of) the conventional numeric (0-9) and related keys (#, *), and other keys used for operating the client computing entity 102 and may include a full set of alphabetic keys or set of keys that may be activated to provide a full set of alphanumeric keys. In addition to providing input, the user input interface can be used, for example, to activate or deactivate certain functions, such as screen savers and/or sleep modes.

The client computing entity 102 can also include volatile storage or memory 322 and/or non-volatile storage or memory 324, which can be embedded and/or may be removable. For example, the non-volatile memory may be ROM, PROM, EPROM, EEPROM, flash memory, MMCs, SD memory cards, Memory Sticks, CBRAM, PRAM, FeRAM, NVRAM, MRAM, RRAM, SONOS, FJG RAM, Millipede memory, racetrack memory, and/or the like. The volatile memory may be RAM, DRAM, SRAM, FPM DRAM, EDO DRAM, SDRAM, DDR SDRAM, DDR2 SDRAM, DDR3 SDRAM, RDRAM, TTRAM, T-RAM, Z-RAM, RIMM, DIMM, SIMM, VRAM, cache memory, register memory, and/or the like. The volatile and non-volatile storage or memory can store databases, database instances, database management systems, data, applications, programs, program modules, scripts, source code, object code, byte code, compiled code, interpreted code, machine code, executable instructions, and/or the like to implement the functions of the client computing entity 102. As indicated, this may include a user application that is resident on the entity or accessible through a browser or other user interface for communicating with the predictive data analysis computing entity 106 and/or various other computing entities.

In another embodiment, the client computing entity 102 may include one or more components or functionality that are the same or similar to those of the predictive data analysis computing entity 106, as described in greater detail above. As will be recognized, these architectures and descriptions are provided for exemplary purposes only and are not limiting to the various embodiments.

In various embodiments, the client computing entity 102 may be embodied as an artificial intelligence (AI) computing entity, such as an Amazon Echo, Amazon Echo Dot, Amazon Show, Google Home, and/or the like. Accordingly, the client computing entity 102 may be configured to provide and/or receive information/data from a user via an input/output mechanism, such as a display, a camera, a speaker, a voice-activated input, and/or the like. In certain embodiments, an AI computing entity may comprise one or more predefined and executable program algorithms stored within an onboard memory storage module, and/or accessible over a network. In various embodiments, the AI computing entity may be configured to retrieve and/or execute one or more of the predefined program algorithms upon the occurrence of a predefined trigger event.

### V. Exemplary System Operations

As discussed in greater detail below, various embodiments of the present invention introduce techniques that improve the training speed of a graph processing machine learning framework given a constant/target predictive accuracy by using a model deficiency data object that is generated for the graph processing machine learning framework using holistic graph links inferred by a graph representation machine learning model. The combination of the noted components enables the proposed graph processing machine learning framework to generate more accurate graph-based predictions, which in turn increases the training speed of the proposed graph processing machine learning framework given a constant predictive accuracy. It is well-understood in the relevant art that there is typically a tradeoff between predictive accuracy and training speed, such that it is trivial to improve training speed by reducing predictive accuracy, and thus the real challenge is to improve training speed without sacrificing predictive accuracy through innovative model architectures. See, e.g., Sun et al., Feature-Frequency—Adaptive On-line Training for Fast and Accurate Natural Language Processing in 40(3) Computational Linguistic 563 at Abst. (“Typically, we need to make a tradeoff between speed and accuracy. It is trivial to improve the training speed via sacrificing accuracy or to improve the accuracy via sacrificing speed. Nevertheless, it is nontrivial to improve the training speed and the accuracy at the same time”). Accordingly, techniques that improve predictive accuracy without harming training speed, such as various techniques described herein, enable improving training speed given a constant predictive accuracy. Therefore, by improving accuracy of performing graph-based machine learning predictions, various embodiments of the present invention improve the training speed of graph processing machine learning frameworks given a constant/target predictive accuracy.

Provided below are exemplary techniques for generating a model deficiency data object and techniques for generating a hybrid risk score. However, while the techniques for generating the model deficiency data object and the techniques for generating a hybrid risk score are described herein as being performed by a single computing entity, a person of ordinary skill in the relevant technology will recognize that each of the noted techniques may be performed by one or more computing entities that may or may not include one or more computing entities used to perform the other set of techniques.

### A. Generating Model Deficiency Data Objects

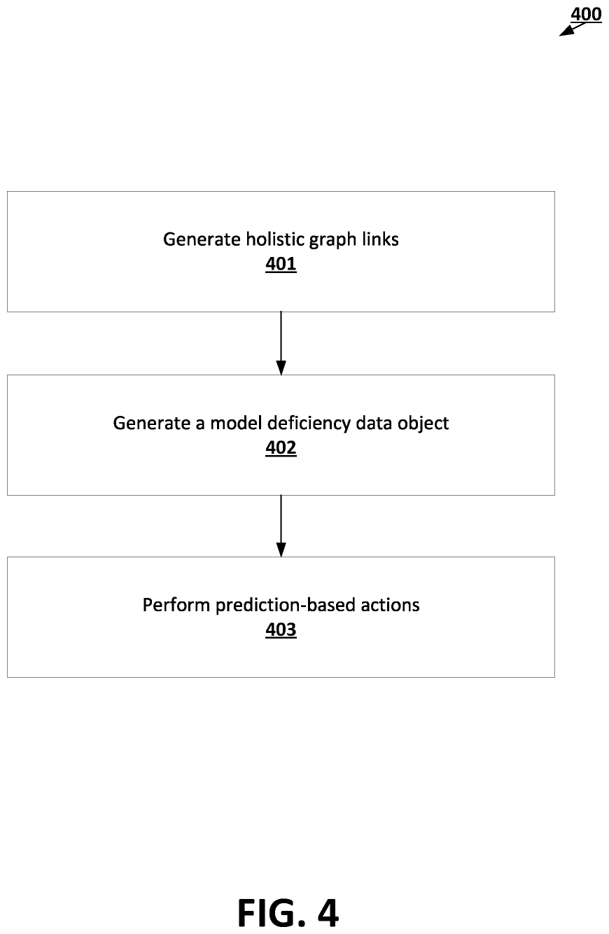

FIG. 4 is a flowchart diagram of an example process 400 for generating a model deficiency data object for a tensor-based graph processing machine learning model that is associated with a risk category. Via the various steps/operations of the process 400, the predictive data analysis computing entity 106 can compare graph links described by tensor-based graph representations that are used to generate input data for a tensor-based graph processing machine learning model with holistic graph representations generated using a graph representation machine learning model to identify deficiency graphs that describe significant predictive relationships inferred across prediction input data that are not captured by the risk tensors used to generate the input data for the tensor-based graph processing machine learning model.

The process 400 begins at step/operation 401 when the predictive data analysis computing entity 106 generates a group of holistic graph links associated with a positive input set for the risk category. In some embodiments, to generate the group of holistic graph links, the predictive data analysis computing entity 106: (i) identifies a positive input set comprising a plurality of prediction input data objects that are assigned an affirmative label defined by the risk category, and (ii) for each prediction input data object in the positive input set, identifies a prediction input feature set describing a group of input features for the prediction input data object.

In some embodiments, a prediction input data object describes a real-world entity and/or a virtual entity with respect to which one or more predictive data analysis operations are performed in order to generate one or more predictive outputs (e.g., a hybrid risk score) for the prediction input data object. An example of a prediction input data object is a patient data object that describes one or more features associated with a particular patient/individual. In some embodiments, features associated with a patient data object include at least one of genomic data associated with the patient data object, behavioral data associated with the patient data object, clinical data associated with the patient data object, demographic data associated with the patient data object, health history data associated with the patient data object, and/or the like. In some embodiments, feature data described by a prediction input data object are referred to herein as the prediction input feature set for the prediction input data object. In some embodiments, each feature described by a prediction input feature set for a prediction input data object belong to an input category that describes a category of heterogenous feature data described by prediction input data objects.

In some embodiments, a risk category describes a label space comprising a set of candidate labels, where the predictive outputs associated with a prediction input data object may be used to assign one candidate label in the label space to the prediction input data object. For example, a particular risk category may be associated with a particular disease/condition and describe a label space comprising a first candidate label that is assigned to a prediction input data object if the prediction input data object is predicted, based at least in part on the predictive outputs (e.g., the hybrid risk score) for the prediction input data object, to be at a high risk of the particular disease/condition and a second candidate label that is assigned to a prediction input data object if the prediction input data object is predicted, based at least in part on the predictive outputs (e.g., the hybrid risk score) for the prediction input data object, to be at a low risk of the particular disease/condition. As another example, a particular risk category may be associated with a particular disease/condition and describe a label space comprising a first candidate label that is assigned to a prediction input data object if the prediction input data object is predicted, based at least in part on the predictive outputs (e.g., the hybrid risk score) for the prediction input data object, to be at a high risk of the particular disease/condition, a second candidate label that is assigned to a prediction input data object if the prediction input data object is predicted, based at least in part on the predictive outputs (e.g., the hybrid risk score) for the prediction input data object, to be at a low risk of the particular disease/condition, and a third candidate label that is assigned to a prediction input data object if the prediction input data object is predicted, based at least in part on the predictive outputs (e.g., the hybrid risk score) for the prediction input data object, to be at a medium risk of the particular disease/condition.

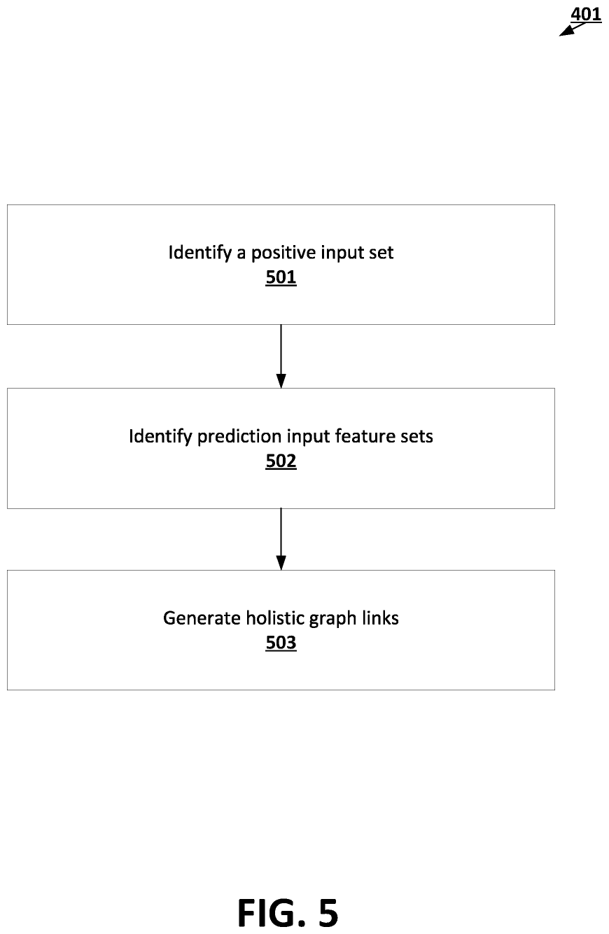

In some embodiments, step/operation 401 may be performed in accordance with the process that is depicted in FIG. 5, which is an example process for generating a group of holistic graph links for a risk category. The process that is depicted in FIG. 5 begins at step/operation 501 when the predictive data analysis computing entity 106 identifies a positive input set for the risk category. A positive input set may comprise a set of prediction input data objects (e.g., a set of patient input data objects) that are associated with an affirmative label defined by a corresponding risk category. For example, the positive input set for a risk category that is associated with a particular disease/condition may include a set of patient data objects associated with a set of patients/individuals that suffer from the particular disease/condition. As another example, the positive input set for a risk category that is associated with a particular disease/condition may include a set of patient data objects associated with a set of patients/individuals that are predicted to be associated with a high risk of developing the particular disease/condition. As described above, in some embodiments, a risk category is associated with a label space defining a set of candidate labels. In some of the noted embodiments, for a given risk category, one of the candidate labels defined by the label space for the given risk category is designated as an affirmative label, and then a set of prediction input data objects whose ground-truth labels correspond to the affirmative label are designated as the prediction input data objects in the positive input sets for the given risk category. For example, when a risk category is associated with a label space for a particular disease/condition that comprises a high-risk label and a low-risk label, the high-risk label may be designated as an affirmative label, and thus those prediction input data objects whose ground-truth labels correspond to the high-risk label may be designated as the positive input set for the noted risk category.

At step/operation 502, the predictive data analysis computing entity 106 identifies, for each prediction input data object in the positive input set, a prediction input feature set. As described above, the prediction input feature set for a prediction input data object may describe one or more features associated with the prediction input data object. For example, the prediction input feature set for a patient data object may describe at least one of genomic features associated with the patient data object, behavioral features associated with the patient data object, clinical features associated with the patient data object, demographic features associated with the patient data object, health history features associated with the patient data object, and/or the like.

At step/operation 503, the predictive data analysis computing entity 106 generates, using a graph representation machine learning model and based at least in part on each prediction input feature set, the group of holistic graph links associated with the risk category. In some embodiments, generating the graph representation machine learning model is configured to extract feature nodes from the prediction input feature sets associated with the positive input set for the risk category, generate co-occurrence edge weights for co-occurrence edges between the feature nodes based at least in part on a frequency of co-occurrence of the corresponding feature in the prediction input feature sets, filter the co-occurrence edges based at least in part on the generated co-occurrence edge weights, and generate the group of holistic links based at least in part on the filtered co-occurrence edges.

In some embodiments, a holistic graph link describes a relationship between two or more features described by the prediction input feature sets for the prediction input data objects in the positive input set for a risk category, where the relationship is generated based at least in part on filtering pairwise relationships between features described by the prediction input feature sets in accordance with predicted relevance measures for the noted pairwise relationships. For example, co-occurrence edge weights generated by a graph representation machine learning model based at least in part on prediction input feature sets for the high-risk population associated with a particular disease/condition may describe that: (i) there is a sufficiently strong correlation between detection of a particular genetic variant in the genomic sequences of the high-risk population and detection of high smoking behavior in the high-risk population, (ii) there is a sufficiently strong correlation between detection of high amounts of usage of a particular inhaler in the high-risk population and detection of the particular genetic variant in the genomic sequences of the high-risk population, and (iii) there is a sufficiently strong correlation between detection of high amounts of usage of the particular inhaler in the high-risk population and detection of high smoking behavior in the high-risk population. In some embodiments, given the noted predictive inferences, a holistic graph link may be generated for the feature set comprising a first feature corresponding to detection of the particular genetic variant, a second feature corresponding to the detection of high smoking behavior, and a third feature corresponding to detection of high amounts of usage of the particular inhaler.

In some embodiments, a holistic graph link associated with H features describes that each feature pair associated with the H features is associated with a co-occurrence edge weight that satisfies (e.g., exceeds) co-occurrence edge weight threshold. For example, consider an exemplary embodiment in which H=2 and the two features comprise a first feature and a second feature. In this example, the holistic graph link associated with the noted feature set describes that the co-occurrence edge weight for a co-occurrence edge between the first feature and the second feature satisfies a co-occurrence edge weight threshold. As another example, consider an exemplary embodiment in which H=3 and the three features comprise a first feature, a second feature, and a third feature. In this example, the holistic graph link associated with the noted feature set describes that: (i) the co-occurrence edge weight for a co-occurrence edge between the first feature and the second feature satisfies a co-occurrence edge weight threshold, (ii) the co-occurrence edge weight for a co-occurrence edge between the first feature and the third feature satisfies the co-occurrence edge weight threshold, and (iii) the co-occurrence edge weight for a co-occurrence edge between the second feature and the third feature satisfies the co-occurrence edge weight threshold.

Accordingly, to generate holistic graph links, it may be important to first generate co-occurrence edge weights for co-occurrence edges between feature nodes, where each feature node is associated with a corresponding feature. In some embodiments, if the prediction input feature sets for a positive input set is associated with a total of F features, then each feature may be associated with a respective feature node to generate a fully connected graph that comprises F feature nodes and

$$\frac{F !}{2 ( F - 2 ) !}$$

co-occurrence edges, with each co-occurrence edge being associated with a distinctive feature pair of

$$\frac{F !}{2 ( F - 2 ) !}$$

distinctive feature pairs. Then, for each co-occurrence edge, a co-occurrence edge weight is generated using a graph representation machine learning model. Afterward, the fully-connected graph is refined by excluding those co-occurrence edges whose corresponding co-occurrence edge weights fail to satisfy (e.g., fail to exceed) a co-occurrence edge weight threshold. This results in a filtered graph having a set of filtered co-occurrence edges who survive the exclusion (i.e., a set of filtered co-occurrence edges whose corresponding co-occurrence edge weights satisfy the co-occurrence edge weight threshold).

In some embodiments, once the filtered graph is generated, the filtered graph is divided into a set of maximally-sized fully-connected subgraphs (i.e., a set of subgraphs each comprising a set of features that have filtered co-occurrence edges between each feature pair in the set, where the set of features is selected such that the addition of any feature into the set causes the resulting subgraph to not be fully connected anymore). In some of the noted embodiments, each maximally-sized fully-connected subgraph that is associated with a respective feature set is used to generate a corresponding holistic graph link that is associated with the noted feature set.

In some embodiments, once the filtered graph is generated, the filtered graph is divided into a set of fully-connected subgraphs (i.e., a set of subgraphs each comprising a set of features that have filtered co-occurrence edges between each feature pair in the set). In some of the noted embodiments, each fully-connected subgraph that is associated with a respective feature set is used to generate a corresponding holistic graph link that is associated with the noted feature set.

For example, consider an operational example in which a positive input set for a risk category comprises three prediction input data objects, where the prediction input feature set for the first prediction input data object in the prediction input set has feature values corresponding to a feature F₁ and a feature F₂, the prediction input feature set for the second prediction input data object in the prediction input set has feature values corresponding to the feature F I, the feature F₂, and a feature F₃, and the prediction input feature set for the third prediction input data object in the prediction input set has feature values corresponding to the feature F₂, the feature F₃, and a feature F₄. In this example, F=4, as the set of features associated with the three prediction input feature sets include the feature F₁, the feature F₂, the feature F₃, and the feature F₄. Given F=4, a fully-connected graph containing

$$\frac{4 !}{2 ( 4 - 2 ) !} = 8$$

co-occurrence edges can be generated, with each co-occurrence edge O_{a,b} being between a feature node a for a feature Fₐ and a feature node b for a feature F_{b}. Suppose in this example the following co-occurrence edges are associated with threshold-satisfying co-occurrence edge weights: O_{1,2}, O_{2,3}, O_{2,4}, and O_{3,4}, while the following co-occurrence edges are associated with non-threshold-satisfying co-occurrence edge weights O_{1,3}, O_{1,4}, O_{2,4}, and O_{3,4}. Accordingly, by excluding the co-occurrence edges O_{1,3}, O_{1,4}, O_{2,4}, and O_{3,4} from the fully-connected graph, a filtered graph comprising the filtered co-occurrence edges O_{1,2}, O_{2,3}, O_{2,4}, and O_{3,4} is generated.

In the example described above, the resulting filtered graph contains two maximally-sized fully-connected subgraphs: a first maximally-sized fully-connected subgraph comprising F₁ and F₂ that is generated based at least in part on the filtered co-occurrence edge O_{1,2}, and a second maximally-sized fully-connected subgraph comprising F₂, F₃, and F₄ that is generated based at least in part on the filtered co-occurrence edges O_{2,3}, O_{2,4}, and O_{3,4}. In some embodiments, two holistic graph links are generated, one corresponding to the first maximally-sized fully-connected subgraph and corresponding to the feature set comprising F₁ and F₂, and the other corresponding to the second maximally-sized fully-connected subgraph and corresponding to the feature set comprising F₂, F₃, and F₄.

Moreover, the resulting filtered graph from the example above contains five fully-connected subgraphs: a first fully-connected subgraph comprising F₁ and F₂ that is generated based at least in part on the filtered co-occurrence edge O_{1,2}, a second fully-connected subgraph comprising F₂, F₃, and F₄ that is generated based at least in part on the filtered co-occurrence edges O_{2,3}, O_{2,4}, and O_{3,4}, a third fully-connected subgraph comprising F₂ and F₃ that is generated based at least in part on the filtered co-occurrence edge O_{2,3}, a fourth fully-connected subgraph comprising F₂ and F₄ that is generated based at least in part on the filtered co-occurrence edge O_{2,4}, and a fifth fully-connected subgraph comprising F₃ and F₄ that is generated based at least in part on the filtered co-occurrence edge O_{3,4}. In some embodiments, three holistic graph links are generated: a first holistic graph link corresponding to the first fully-connected subgraph and corresponding to the feature set comprising F₁ and F₂, a second holistic graph link corresponding to the second fully-connected subgraph and corresponding to the feature set comprising F₂, F₃, and F₄, a third holistic graph link corresponding to the third fully-connected subgraph and corresponding to the feature set comprising F₂ and F₃, a fourth holistic graph link corresponding to the fourth fully-connected subgraph and corresponding to the feature set comprising F₂ and F 4, and a fifth holistic graph link corresponding to the fifth fully-connected subgraph and corresponding to the feature set comprising F₃ and F₄.

In some embodiments, to generate the co-occurrence edge weight for a co-occurrence edge between a first feature node associated with a first feature and a second feature node associated with a second feature, a graph representation machine learning model is utilized. A graph representation machine learning model may be configured to generate a co-occurrence edge weight for a co-occurrence edge between two node features. In some embodiments, given F features and P prediction input feature sets that are associated with the positive input set for a risk category, to generate the co-occurrence edge weight for a co-occurrence edge between a first feature node associated with a first feature and a second feature node associated with a second feature, a graph representation machine learning model first generates P per-input co-occurrence likelihood values for the feature pair comprising the first feature and the second feature, with each per-input co-occurrence likelihood value being associated with a corresponding prediction input feature set in P prediction input feature sets that are associated with the positive input set and describing a predicted likelihood that the prediction input feature set describes occurrence/detection of both the first feature and the second feature. Then, to generate the co-occurrence edge weight for the noted co-occurrence edge, the graph representation machine learning model: (i) aggregates all of the P per-input co-occurrence likelihood values for the feature pair comprising the first feature and the second feature to generate a cross-input co-occurrence likelihood value, and (ii) normalizes the

$$\frac{F !}{2 ( F - 2 ) !}$$

cross-input co-occurrence likelihood values across the

$$\frac{F !}{2 ( F - 2 ) !}$$

feature pairs to generate

$$\frac{F !}{2 ( F - 2 ) !}$$

co-occurrence edge weights for the

$$\frac{F !}{2 ( F - 2 ) !}$$

co-occurrence edges.

For example, consider an exemplary embodiment in which F=3 and P=2, where L_{a,b,c} is the per-input co-occurrence likelihood value for the feature pair comprising Fₐ and F_{b} in the cth prediction input feature set, and LL_{a,b} is the cross-input co-occurrence likelihood value for the feature pair comprising Fₐ and F_{b} that can be calculated using the equation LL_{a,b}=ΣPᵢ₌₁^{P}L_{a,b,i}. In this example,

$$\frac{F !}{2 ( F - 2 ) !} = 3$$

cross-input co-occurrence likelihood values may be generated and normalized (e.g., using softmax normalization) to generate the

$$\frac{F !}{2 ( F - 2 ) !} = 3$$

co-occurrence edge weights for

$$\frac{F !}{2 ( F - 2 ) !} = 3$$

co-occurrence edges (i.e., the co-occurrence edge weight for the co-occurrence edge between the feature node for feature F₁ and the feature node for feature F₂, the co-occurrence edge weight for the co-occurrence edge between the feature node for feature F₁ and the feature node for feature F₃, and the co-occurrence edge weight for the co-occurrence edge between the feature node for feature F₂ and the feature node for feature F₃).

In some embodiments, given F features and P prediction input feature sets that are associated with the positive input set for a risk category, to generate the co-occurrence edge weight for a co-occurrence edge between a first feature node associated with a first feature and a second feature node associated with a second feature, a graph representation machine learning model first generates P per-input co-occurrence likelihood values for the feature pair comprising the first feature and the second feature, with each per-input co-occurrence likelihood value being associated with a corresponding prediction input feature set in P prediction input feature sets that are associated with the positive input set and describing a predicted likelihood that the prediction input feature set describes occurrence/detection of both the first feature and the second feature. Then, to generate the co-occurrence edge weight for the noted co-occurrence edge, the graph representational learning machine learning model: (i) divides the P per-input co-occurrence likelihood values for the feature pair comprising the first feature and the second feature into a first subset that satisfies a per-input co-occurrence likelihood value threshold and a second subset that fails to satisfy the per-input co-occurrence likelihood value threshold, (ii) generates an affirmative cross-input co-occurrence likelihood value by combining the P per-input co-occurrence likelihood values in the first subset, (iii) generates a negative cross-input co-occurrence likelihood value by combining the P per-input co-occurrence likelihood values in the second subset, and (iv) generates the co-occurrence edge weight for the noted co-occurrence edge based at least in part on a measure of deviation between the affirmative cross-input co-occurrence likelihood value and the negative cross-input co-occurrence likelihood value.

For example, consider an exemplary embodiment in which F=3 and P=2, where L_{a,b,c} is the per-input co-occurrence likelihood value for the feature pair comprising Fₐ and F_{b} in the cth prediction input feature set. In this example, if L_{1,2,1}=0.3, L_{1,2,2}=0.6, L_{1,3,1}=0.2, L_{1,3,2}=0.8, L_{2,3,1}=0.9, L_{2,3,2}=0.7, and if the per-input co-occurrence likelihood value is 0.5, then the co-occurrence edge weight for the co-occurrence edge associated with F₁ and F₂ may be determined based at least in part on 0.6-0.3, the co-occurrence edge weight for the co-occurrence edge associated with F₁ and F₃ may be determined based at least in part on 0.8-0.2, and the co-occurrence edge weight for the co-occurrence edge associated with F₁ and F₃ may be determined based at least in part on 0.9+0.7−0.

In some embodiments, the graph representation machine learning model uses at least one of the techniques described in Hamilton, Graph Representation Learning, in Synthesis Lectures on Artificial Intelligence and Machine Learning, available online at https://doi.org/10.2200/S01045ED 1 V01Y202009AIM046 (2020). In some embodiments, given F features, the graph representational machine learning model comprises an embedding machine learning model and

$$\frac{F !}{2 ( F - 2 ) !}$$

co-occurrence detection machine learning models, where each co-occurrence detection machine learning model is associated with a corresponding distinctive feature pair and generates a per-input co-occurrence likelihood value for a given prediction input feature set and the corresponding distinctive feature pair that is associated with the co-occurrence detection machine learning model. In some embodiments, for a given prediction input feature set of P prediction input feature sets, the embedding machine learning model (e.g., an attention-based text encoder machine learning model) processes the given prediction input feature set (e.g., sequential text data associated with the given prediction input feature set) to generate a prediction input feature set embedding, and then each of the

$$\frac{F !}{2 ( F - 2 ) !}$$

co-occurrence detection machine learning models process the prediction input feature set embedding to generate the per-input co-occurrence likelihood value for the given prediction input feature set and the corresponding distinctive feature pair for the particular co-occurrence detection machine learning models. For example, given F=3, then six co-occurrence detection machine learning models may be generated, where each co-occurrence detection machine learning model may be associated with a feature pair comprising a feature Fₐ and F_{b} and may be configured to process the prediction input feature set embedding for a particular prediction input feature set to generate a per-input co-occurrence likelihood value for the feature pair and the particular prediction input feature set.

In some embodiments, for a given prediction input feature set of P prediction input feature sets, the embedding machine learning model (e.g., an attention-based text encoder machine learning model) processes the P prediction input feature sets to generate P prediction input feature set embeddings. Then, each co-occurrence detection machine learning model that is associated with a particular distinctive feature pair processes the prediction input feature set embedding for the given prediction input feature set as a primary input and the P−1 prediction input feature set embeddings for those prediction input feature sets other than the given prediction input feature set as the auxiliary inputs using a cross-input attention mechanism (e.g., a cross-input bidirectional attention mechanism) to generate the per-input co-occurrence likelihood value for the given prediction input feature set and the corresponding distinctive feature pair for the particular co-occurrence detection machine learning model. For example, given F=3, then six co-occurrence detection machine learning models may be generated, where each co-occurrence detection machine learning model may be associated with a feature pair comprising a feature Fₐ and F_{b} and may be configured to process the prediction input feature set embedding for a particular prediction input feature set as the primary inputs and the prediction input feature set embeddings for the other prediction input features as auxiliary inputs to generate a per-input co-occurrence likelihood value for the feature pair and the particular prediction input feature set.

In some embodiments, inputs to the graph representation machine learning model comprise T vectors, where T may define the number of input tokens generated based at least in part on an input prediction input feature set or the number of input tokens generated based at least in part on all of the prediction input feature sets depending on the embodiments. In some embodiments, outputs of the graph representation machine learning model comprise a vector describing

$$\frac{F !}{2 ( F - 2 ) !}$$

per-input co-occurrence likelihood values for an input prediction input feature set across

$$\frac{F !}{2 ( F - 2 ) !}$$

distinctive feature pairs for F features in a relevant feature space. In some embodiments, the graph representation machine learning model is defined using training data describing ground-truth observations about co-occurrence of features in particular real-world entities and/or particular virtual-world entities (e.g., ground-truth observations about co-occurrence of features across input categories in a particular patient data object).

In some embodiments, an input category describes a defined attribute of a feature in a prediction input feature set for a prediction input data object that is used to divide the prediction input feature set into categorical subsets that are in turn used to generate risk tensors. Examples of input categories for the prediction input feature set for a patient input data object include at least one of a genomic data input category, a behavioral data risk category, a clinical data risk category, a demographic data risk category, a health history data risk category, and/or the like. In some embodiments, given a positive input set for a risk category that comprises P prediction input data objects (e.g., corresponding to P patients/individuals that suffer from a particular disease/condition), P prediction input feature sets are identified, where each prediction input feature set comprises the prediction input features for a corresponding prediction input data object. Importantly, in some embodiments, the prediction input features for a particular prediction input data object comprises features associated with C input categories. In some of the noted embodiments, holistic graph links are generated based at least in part on co-occurrence edge weights that are generated based at least in part on the totality of each of the P prediction input feature sets, which include each feature described by the P prediction input feature sets regardless of input category. In other words, if S_{a,b} describes features of an ath prediction input data object that belong to a bth input category, co-occurrence edge weights that are used to generate holistic graph links are generated based at least in part on a feature set that comprises Σᵢ₌₁^{P}Uⱼ₌₁^{C}S_{i,j}. This is an important property, because it means that holistic graph links are able to capture predictive associations across prediction input data objects as well as predictive associations across input categories, which means that the holistic graph links can include predictive insights that are not captured by tensor-based graph representations used to generate input data for a particular prediction input data that is provided to a tensor-based graph processing machine learning model.

Returning to FIG. 4, at step/operation 402, the predictive data analysis computing entity 106 generates a model deficiency data object based at least in part on the group of holistic graph links. In some embodiments, the model deficiency data object describes at least a subset (e.g., a ranked subset of) those holistic graph links that are not present in the tensor-based graph links associated with a tensor-based graph processing machine learning model. In some embodiments, the model deficiency data object comprises a selected subset of the one or more deficiency graph links that is generated based at least in part on: (i) an immutability score for each deficiency graph link, (ii) an actionability score for each deficiency graph link, and (iii) a prevalence score for each deficiency graph link.

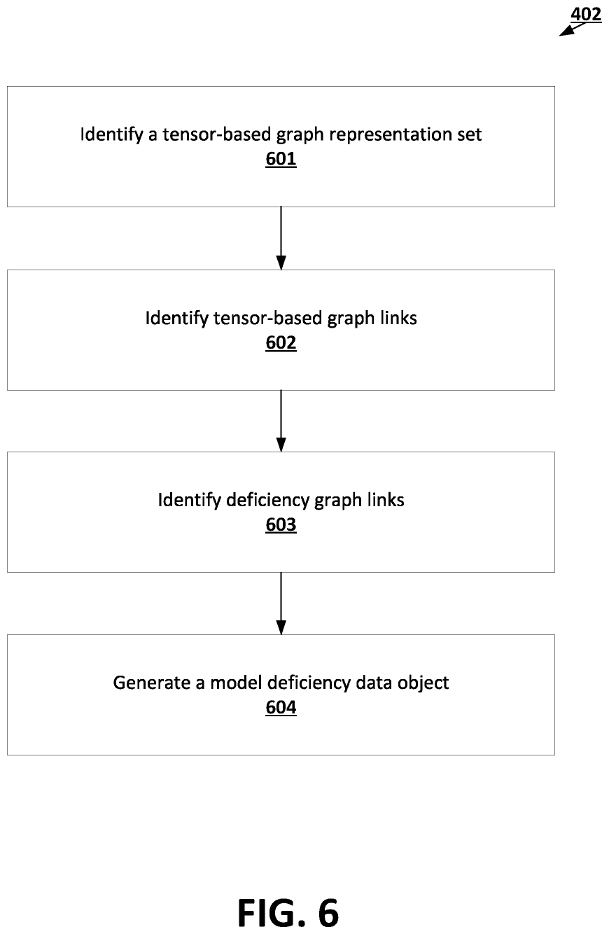

In some embodiments, step/operation 402 may be performed in accordance with the process that is depicted in FIG. 6, which is an example process for generating a model deficiency data object for a risk category. The process that is depicted in FIG. 6 begins at step/operation 601 when the predictive data analysis computing entity 106 identifies a tensor-based graph representation set for the risk category that comprises the tensor-based graph representations for each prediction input data object that is in the positive input set for the risk category.

In some embodiments, each prediction input data object of P prediction input data objects in the positive input set for a risk category is associated with a prediction input feature set that comprises features associated with C input categories. Accordingly, for a given prediction input data object that is associated with a given prediction input feature set, the given prediction input feature set can be divided into C categorical subsets, where each categorical subset comprises a subset of the given prediction input feature set that is associated with a corresponding category of C input categories. In some embodiments, each categorical subset of the C categorical subsets for the given prediction input data object is used to generate a risk tensor of C risk tensors, with each risk tensor being associated with an input category of C input categories and being generated based at least in part on the categorical subset for the corresponding input category. In some embodiments, each risk tensor is used to generate a tensor-based graph representation that is a graph-based representation of the risk tensor, such that the given prediction input data object is associated with C tensor-based graph representations. In some embodiments, each tensor-based graph representation is processed using a graph embedding machine learning model (e.g., a Node2Vec-based graph embedding machine learning model) to generate a tensor-based graph feature embedding, such that the given prediction input data object is associated with C graph-based feature embeddings. In some embodiments, a tensor-based graph processing machine learning model is configured to process C graph-based feature embeddings for the given prediction input data object to generate an inferred hybrid risk score for the given prediction input data object. Exemplary embodiments of risk tensors, tensor-based graph representations, tensor-based graph feature embeddings, and tensor-based graph processing machine learning models are described in Subsection B of the present Section IV of the present document.

In some embodiments, given a risk category that is associated with P prediction input data objects (e.g., a disease/condition that is associated with P afflicted patient data objects, a disease/condition that is associated with P high-risk patient data objects, and/or the like), and given C input categories (e.g., given C=2 input categories comprising a genomic data input category and a clinical data input category), P*C categorical subsets of the P prediction input feature sets for the P prediction input data objects can be identified/generated, where the P*C categorical subsets can be used to generate P*C risk tensors, the P*C risk tensors can be used to generate P*C tensor-based graph representations, and the P*C tensor-based graph representations can be used to generate P*C tensor-based graph feature embeddings. In some embodiments, the P*C tensor-based graph representations are hereby referred to as the tensor-based graph representation set for the risk category.

At step/operation 602, the predictive data analysis computing entity 106 identifies a group of tensor-based graph links that are in at least one of the tensor-based graph representations in the tensor-based graph representation set for the risk category. In some embodiments, a tensor-based graph link describes a feature set whose collective relationship is captured by at least one tensor-based graph representation in the tensor-based graph representation set for a corresponding risk category. In some embodiments, because at least one tensor-based graph representation in the tensor-based graph representation set for a corresponding risk category captures the collective relationship associated with the feature set, the predictive data analysis computing entity 106 infers that the collective relationship is captured by the risk tensors used to generate input data for a tensor-based graph processing machine learning model that is associated with the corresponding risk category, and thus the tensor-based graph processing machine learning model that is associated with the corresponding risk category is not deficient with respect to the noted collective relationship.

In some embodiments, a tensor-based graph link describes a maximally-sized fully-connected subgraph of a tensor-based graph representation in the tensor-based graph representation set for a corresponding risk category. For example, consider an exemplary embodiment in which the set of tensor-based graph edges described by the tensor-based graph representations in the tensor-based graph representation set for a corresponding risk category includes O_{1,2}, O_{1,3}, O_{2,3}, and O_{3,4}. In this example, the tensor-based graph representation set is associated with a first tensor-based graph link that is associated with the features F₁, F₂, and F₃, and a second tensor-based graph link that is associated with the features F₃ and F₄.

In some embodiments, a tensor-based graph link describes a fully-connected subgraph of a tensor-based graph representation in the tensor-based graph representation set for a corresponding risk category. For example, consider an exemplary embodiment in which the set of tensor-based graph edges described by the tensor-based graph representations in in the tensor-based graph representation set for a corresponding risk category includes O_{1,2}, O_{1,3}, O_{2,3}, and O_{3,4}. In this example, the tensor-based graph representation set is associated with a first tensor-based graph link that is associated with the features F₁, F₂, and F₃, a second tensor-based graph link that is associated with the features F₃ and F₄, a third tensor-based graph link that is associated with the features F₁ and F₂, a fourth tensor-based graph link that is associated with the features F₁ and F₃, and a fifth tensor-based graph link that is associated with the features F₂ and F₃.

At step/operation 603, the predictive data analysis computing entity 106 generates/identifies one or more deficiency graph links that are in the group of holistic graph links but are not in the group of tensor-based graph links. In some embodiments, because a deficiency graph link is in the group of holistic graph links generated based at least in part on holistic processing of prediction input feature sets across all of the P prediction input data objects in the positive input set for the risk category and across all of the input categories, but not in the group of tensor-based graph links that are mapped to an input space of a tensor-based graph machine learning model, the tensor-based graph machine learning model is predicted to be deficient with respect to the deficiency graph link, such that the input mechanism of the tensor-based graph machine learning model is not predicted to capture predictive insights related to presence of feature data corresponding to the deficiency graph link.

At step/operation 604, the predictive data analysis computing entity 106 generates the model deficiency data object based at least in part on the one or more deficiency graph links. In some embodiments, to generate the model deficiency data object, the predictive data analysis computing entity 106: (i) ranks the one or more deficiency graph links, (ii) selects the top-ranked N deficiency graph links, (iii) generates a prevalent subset of the selected deficiency graph links whose prevalence scores satisfies a prevalence score threshold, (iv) generates a non-action-related subset of the prevalent subset that are not associated with an existing predictive action, and (v) generates the model deficiency data object based at least in part on (e.g., to comprise) data associated with the deficiency graph links that fall within the non-action-related subset.

In some embodiments, ranking the deficiency graph links is performed in a descending manner and based at least in part on ranking measures for the deficiency graph links, where the ranking measure for a deficiency graph link is determined based at least in part on at least one of an immutability score for the deficiency graph link and an actionability score for the deficiency graph link. In some embodiments, the immutability score for a deficiency graph link describes how much the feature set for the deficiency graph link is immutable, such that, for example, a feature set that includes a genetic/genomic feature may be determined to be more immutable than a feature set that includes a behavioral feature. In some embodiments, the actionability score for a deficiency graph link describes an estimated cost/feasibility measure for a predictive action (e.g., a clinical trial action) that is configured to establish a ground-truth/observed correlation measure for the feature set that is associated with the deficiency graph link.

In some embodiments, the prevalence score for a deficiency graph link describes an estimated measure of the ratio of a predictive input population (e.g., a patient/individual population) that has features corresponding to the feature set for the deficiency graph link to a total predictive input population. In some embodiments, the prevalence score for a deficiency graph link describes whether the feature set associated with the deficiency graph link is present in a statistically significant portion of the population. In some embodiments, by factoring in the rarity of the disease (e.g., as calculated via analysis of clinical research) associated with the risk category, the predictive data analysis computing entity 106 determines if the risk factors associated with the feature set for a deficiency graph link are present in a statistically significant portion of the population. This is a critical step as selecting a smaller population size may be feasible for a rare disease, but a more common disease would need a much larger population size to ensure that it was representative of the general population for the disease in question. If multiple risk factors are found to be present in a statistically significant portion of the population, then those risk factors could be studied as a group rather than individually. This would aid in the understanding of complex diseases that have multiple genetic variants that influence their severity and treatment, such as the dystrophy gene. In some embodiments, if an identified risk factor is suspected to be of significance by either manual research or the steps described above but falls short of statistical significance for the population, that risk factor can be revisited at a later date to ensure that the further analysis would be meaningful.

In some embodiments, the non-action-related subset of the prevalent subset of the deficiency graph links describes each deficiency graph link that is both associated with a threshold-satisfying prevalence score (e.g., whose prevalence score describes that the feature set for the deficiency graph link is present in a statistically significant portion of the population), and that are not associated with an existing predictive action such as a clinical trial that is configured to establish a ground-truth/observed correlation measure for the feature set that is associated with the deficiency graph link. In some embodiments, if a statistically significant number of individuals are present with the risk factors in question, the predictive data analysis computing entity 106 analyzes public domain sources (e.g., https://clinicaltrials.gov/ct2/home) to determine if any clinical trials exist for the specific risk factors and the diseases in question. In some embodiments, if no clinical trials exist, then one could be created internally utilizing the care delivery network, or the data generated around the diseases and the risk factors as well as the population could be handed off to a partner organization to run the trial, or the information generated could be licensed to life science organizations for a monetary fee, then the corresponding deficiency graph link is added to the model deficiency data object. In some embodiments, if clinical trials exist, and are actively running, the data generated around the risk factor(s) and the identified population could be flagged. The entity running the clinical could then be contacted about utilizing the flagged population as part of their ongoing trial.

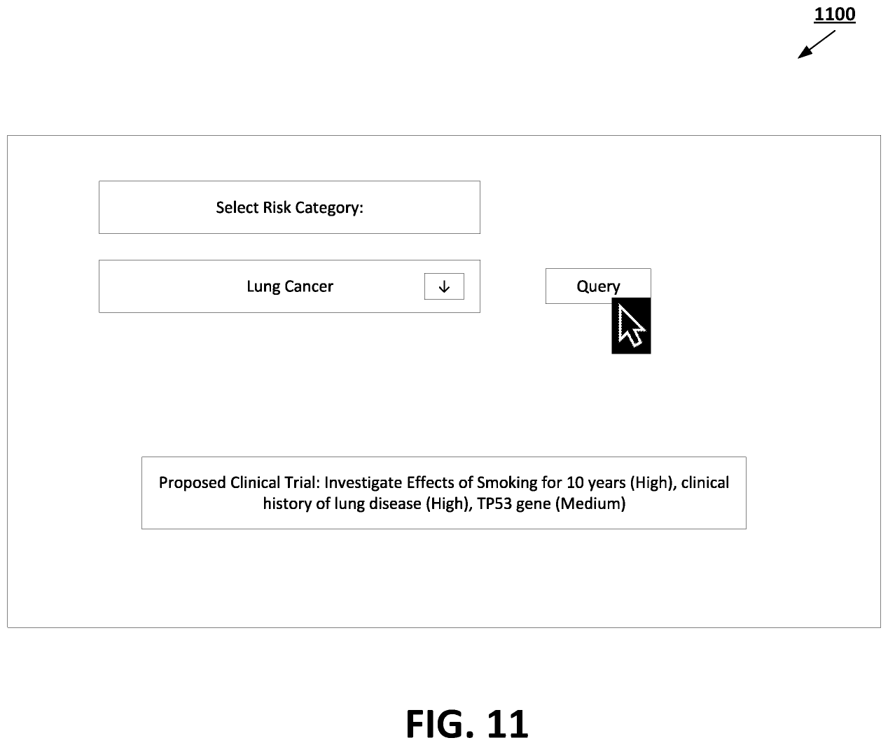

Returning to FIG. 4, at step/operation 403, the predictive data analysis computing entity 106 performs one or more prediction-based actions based at least in part on the model deficiency data object. In some embodiments, performing the prediction-based actions comprises generating user interface data for a prediction output user interface that describes a model deficiency data object and/or recommended predictive actions that are determined based at least in part on the model deficiency data object. An operational example of such a prediction output user interface 1100 is depicted in FIG. 11.

An example of a prediction-based action that may be performed at step/operation 403 relates to performing operational load balancing for post-prediction systems that perform post-prediction operations (e.g., automated specialist appointment scheduling operations) based at least in part on graph-based predictions. For example, in some embodiments, a predictive recommendation computing entity determines D classifications for D prediction input data objects using a graph processing machine learning framework that is augmented by a model deficiency data object that is generated for the graph processing machine learning framework using holistic graph links inferred by a graph representation machine learning model. Then, the count of D prediction input data objects that are associated with an affirmative classification, along with a resource utilization ratio for each prediction input data object, can be used to predict a predicted number of computing entities needed to perform post-prediction processing operations with respect to the D prediction input data objects. For example, in some embodiments, the number of computing entities needed to perform post-prediction processing operations (e.g., automated specialist scheduling operations) with respect to D prediction input data objects can be determined based at least in part on the output of the equation: R=ceil(Σₖ^{k=K} urₖ), where R is the predicted number of computing entities needed to perform post-prediction processing operations with respect to the D prediction input data objects, ceil(.) is a ceiling function that returns the closest integer that is greater than or equal to the value provided as the input parameter of the ceiling function, k is an index variable that iterates over K prediction input data objects among the D prediction input data objects that are associated with affirmative classifications, and urₖ is the estimated resource utilization ratio for a kth prediction input data object that may be determined based at least in part on a patient history complexity of a patient associated with the prediction input data object. In some embodiments, once R is generated, a predictive recommendation computing entity can use R to perform operational load balancing for a server system that is configured to perform post-prediction processing operations with respect to D prediction input data objects. This may be done by allocating computing entities to the post-prediction processing operations if the number of currently-allocated computing entities is below R, and deallocating currently-allocated computing entities if the number of currently-allocated computing entities is above R.

Accordingly, as discussed in greater detail above, various embodiments of the present invention introduce techniques that improve the training speed of a graph processing machine learning framework given a constant/target predictive accuracy by using a model deficiency data object that is generated for the graph processing machine learning framework using holistic graph links inferred by a graph representation machine learning model. The combination of the noted components enables the proposed graph processing machine learning framework to generate more accurate graph-based predictions, which in turn increases the training speed of the proposed graph processing machine learning framework given a constant predictive accuracy. It is well-understood in the relevant art that there is typically a tradeoff between predictive accuracy and training speed, such that it is trivial to improve training speed by reducing predictive accuracy, and thus the real challenge is to improve training speed without sacrificing predictive accuracy through innovative model architectures. See, e.g., Sun et al., Feature-Frequency—Adaptive On-line Training for Fast and Accurate Natural Language Processing in 40(3) Computational Linguistic 563 at Abst. (“Typically, we need to make a tradeoff between speed and accuracy. It is trivial to improve the training speed via sacrificing accuracy or to improve the accuracy via sacrificing speed. Nevertheless, it is nontrivial to improve the training speed and the accuracy at the same time”). Accordingly, techniques that improve predictive accuracy without harming training speed, such as various techniques described herein, enable improving training speed given a constant predictive accuracy. Therefore, by improving accuracy of performing graph-based machine learning predictions, various embodiments of the present invention improve the training speed of graph processing machine learning frameworks given a constant/target predictive accuracy.

### B. Generating Hybrid Risk Scores

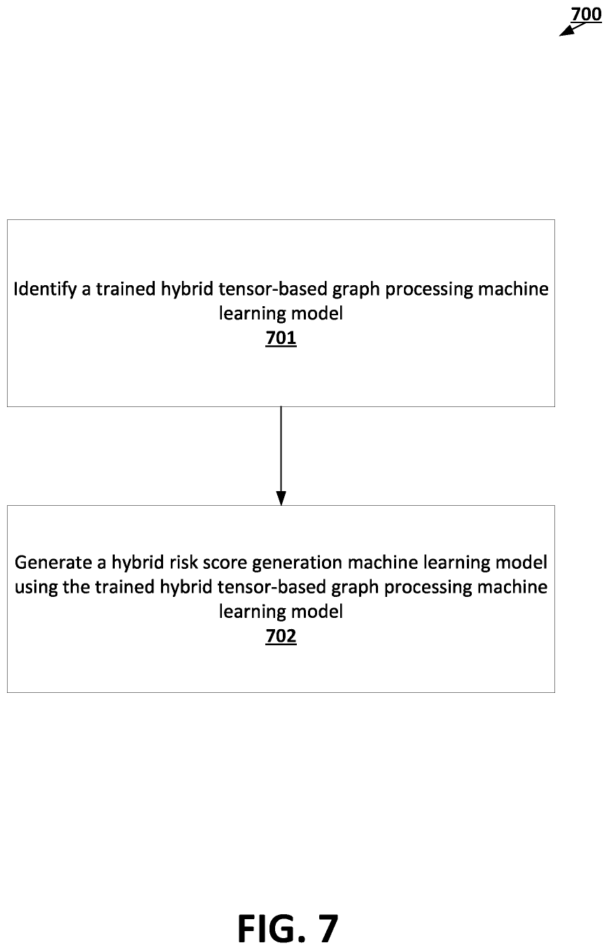

FIG. 7 is a flowchart diagram of an example process 700 for generating a hybrid risk score generation machine learning model. Via the various steps/operations of the process 700, the predictive data analysis computing entity 106 can generate a model that is configured to enable efficient hybrid risk score generation using a fewer number of preconfigured parameters relative to various conventional deep learning models.

The process 700 begins at step/operation 701 when the predictive data analysis computing entity 106 identifies a trained tensor-based graph processing machine learning model. In some embodiments, the predictive data analysis computing entity 106 retrieves configuration data (e.g., parameter data, hyper-parameter data, and/or the like) for the trained tensor-based graph processing machine learning model from the storage subsystem 108. In some embodiments, the predictive data analysis computing entity 106 performs one or more model training operations to generate configuration data (e.g., parameter data, hyper-parameter data, and/or the like) for the trained tensor-based graph processing machine learning model.

The trained tensor-based graph processing machine learning model is a trained machine learning model that is configured to process one or more tensor-based graph feature embeddings for a prediction input data object in order to generate an inferred hybrid risk score for the prediction input data object. The trained tensor-based graph processing machine learning model may be configured to receive, as at least a part of its inputs, one or more tensor-based graph feature embeddings for a prediction input data object, where a tensor-based graph feature embedding may be a vector of one or more values that are determined based at least in part on a tensor-based graph representation of a risk tensor of one or more risk tensors for the prediction input data object. In some embodiments, the trained tensor-based graph processing machine learning model may include a plurality of graph-based machine learning models (e.g., including one or more graph convolutional neural network machine learning models), where each graph-based machine learning model is configured to process a tensor-based graph feature embedding for a particular input category to generate a per-model machine learning output, and an ensemble machine learning model that is configured to aggregate/combine per-model machine learning outputs across various graph-based machine learning models to generate the inferred hybrid risk score for the prediction input data object. For example, a trained tensor-based graph processing machine learning model may include: a first graph-based machine learning model that is configured to process a tensor-based graph feature embedding determined based at least in part on genomic data (e.g., based at least in part on a genomic risk tensor) for a prediction input data object in order to generate a first per-model machine learning output, a second graph-based machine learning model that is configured to process a tensor-based graph feature embedding determined based at least in part on clinical data (e.g., based at least in part on a clinical risk tensor) for a prediction input data object in order to generate a second per-model machine learning output, a third graph-based machine learning model that is configured to process a tensor-based graph feature embedding determined based at least in part on behavioral data (e.g., based at least in part on a behavioral risk tensor) for a prediction input data object in order to generate a third per-model machine learning output, and an ensemble machine learning model that is configured to aggregate/combine the first per-model machine learning output, the second per-model machine learning output, and the third per-model machine learning output in order to generate the inferred hybrid risk score for the prediction input data object. In some embodiments, the trained tensor-based graph processing machine learning model is trained (e.g., via one or more end-to-end training operations) using ground-truth hybrid risk scores for a set of ground-truth tensor-based graph feature embeddings for each training prediction input data object of a set of training prediction input data objects.

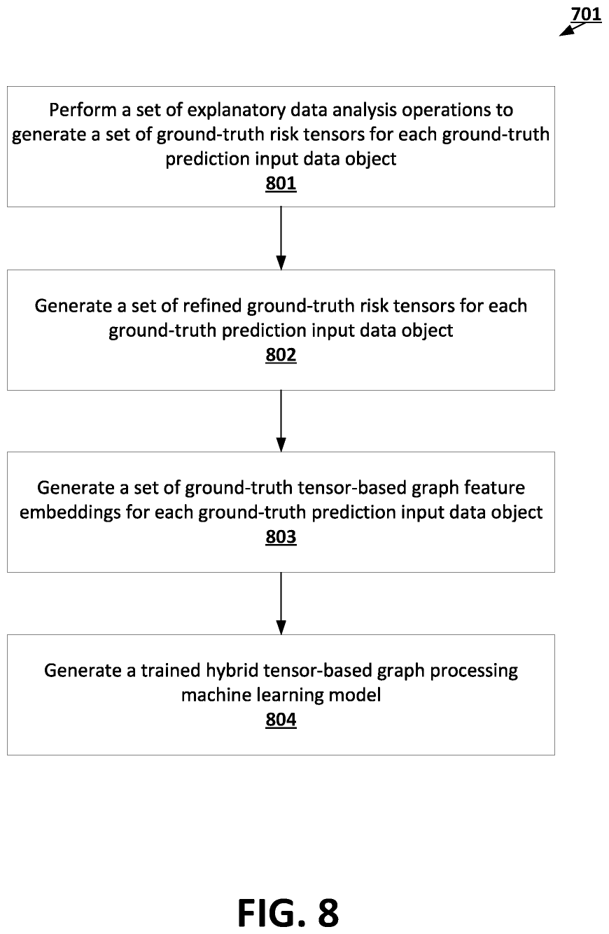

In some embodiments, performing step/operation 701 includes generating the trained tensor-based graph processing machine learning model, for example in accordance with the process that is depicted in FIG. 8. The process that is depicted in FIG. 8 begins at step/operation 801, when the predictive data analysis computing entity 106 performs a set of explanatory data analysis operations on predictive input feature set data associated with a set of ground-truth prediction input data objects to generate, for each ground-truth prediction input data object of the set of ground-truth prediction input data objects, a set of ground-truth risk tensors. In some embodiments, the predictive data analysis computing entity 106 performs explanatory data analysis on relevant data sets associated with a prediction input data object to determine a set of data inferences, such as clinical inferences, genomic inferences, medication inferences, treatment inferences, electronic medical record (EMR) inferences, claims-related inferences, social health determinant inferences, patient-reported outcome inferences, primary care inferences, medical image inferences, and/or the like. In some embodiments, the data inferences are used to determine ground-truth risk tensors.

In general, a risk tensor may describe a tensor data object that includes a set of subject-matter-defined data items associated with a prediction input data object. In some embodiments, a risk tensor describes a heterogeneous group of data that are related by the underlying risk tensor and relate to an input category that corresponds to the risk tensor. Examples of risk tensors include a genomic risk tensor that includes genomic data associated with a prediction input data object, a behavioral risk tensor that includes behavioral data associated with a prediction input data object, a clinical risk tensor that includes clinical data associated with a prediction input data object, a demographic risk tensor that includes demographic data associated with a prediction input data object, a health history risk tensor that includes health history data associated with a prediction input data object, and/or the like.

For example, a genomic risk tensor may describe at least one of ribonucleic acid (RNA)-seq data, complex molecular biomarkers relating to oncology (e.g. tumor mutational burden), single nucleotide polymorphisms, deoxyribonucleic acid (DNA) methylation data from panels, and/or the like. In some embodiments, all of the noted data items may be grouped, because they are all related to the genomic risk profile for a given disease, even though the data items are different, relate to different aspects of the genome, and come in different file formats (e.g. FASTQ files for DNA data, .IDAT files for Illumina Infinium HumanMethylation480 chip data, and/or the like). In some embodiments, if specific data items are assumed, from existing clinical practice, to be highly influential (e.g. smoking status and pack history for lung cancer), then a confidence score in the relevance of each risk tensor, and the necessary volumes of data, may be generated. The output from the confidence score can be used to assess whether there are sufficient data to adopt the risk tensor data, or whether additional data are recommended (e.g. data augmentation is needed in the case of medical image data). In some embodiments, when a risk tensor is associated with a ground-truth inferred hybrid risk score and used to generate a trained tensor-based graph processing machine learning model (e.g., by generating ground-truth tensor-based graph feature embeddings that are used as inputs during the training of a tensor-based graph processing machine learning model), the risk tensor is referred to as a ground-truth risk tensor. In some embodiments, when a risk tensor is used to generate inferred hybrid risk scores that are in turn used to generate a hybrid risk score generation machine learning model, the risk tensor is referred to as a prior risk tensor.

At step/operation 802, the predictive data analysis computing entity 106 generates, for each ground-truth prediction input data object of the set of ground-truth prediction input data objects, a set of refined ground-truth risk tensors based at least in part on the set of ground-truth risk tensors for the ground-truth prediction input data object. However, while various embodiments of the present invention describe refining risk ground-truth tensors to generate refined ground-truth risk tensors, a person of ordinary skill in the relevant technology will recognize that in some embodiments ground-truth risk tensors may be automatically adopted as refined risk tensors and thus refinement operations may be skipped.

In some embodiments, to perform step/operation 802, the predictive data analysis computing entity 106 generates a confidence score for the constituent data in each particular ground-truth risk tensor. The confidence score for a ground-truth risk score may be generated based at least in part on the volume of the constituent data, the estimated magnitude of the signal strength or statistical power of a given feature (e.g. Cohen's d score for effect size) of the constituent data, constituent data completeness, and quality and the presence of redundant and highly-correlated genomic variants and collinear features among the constituent data. In some embodiments, if the confidence score for a given ground-truth risk tensor fails to satisfy a confidence score threshold (e.g., falls short of the minimum threshold score for adaptation by the predictive data analysis computing entity 106 as, for example, determined by expert data scientists), then the failure is flagged and the predictive data analysis computing entity 106 will attempt automatically to source new data. There are several different ways that this confidence thresholding technique could be performed, depending upon the specific type of the ground-truth risk tensor. For example, if the confidence score was low for a genomic risk tensor, the predictive data analysis computing entity 106 may interact with the application programming interfaces (APIs) of public-domain genomics data repositories, such as the Sequence Read Archive (available online at https://www.ncbi.nlm.nih.gov/sra), to acquire supplementary data. For a clinical risk tensor, the predictive data analysis computing entity 106 may query suitable EMR data sources for more historical data, or perform data augmentation on medical image data. This process may continue until a sufficient threshold confidence score is reached (at which point the latest state of a ground-truth risk tensor may be adopted as a refined ground-truth risk tensor), or, as a last resort, a notification to a human operator is transmitted after a defined number of tensor augmentation operations are performed.

At step/operation 803, the predictive data analysis computing entity 106 generates, for each ground-truth prediction input data object of the set of ground-truth prediction input data objects, a set of ground-truth tensor-based graph feature embeddings based at least in part on the set of refined ground-truth risk scores for the ground-truth prediction input data object. In some embodiments, to perform step/operation 803, the predictive data analysis computing entity 106 performs at least one of the following on each set of refined ground-truth risk tensors for a ground-truth patient: one or more data quality operations, one or more data engineering operations, one or more first-order feature engineering operations, and/or the like.

A tensor-based graph feature embedding may be a fixed-size representation of a tensor-based graph representation, which is a graph-based representation of a risk tensor. In some embodiments, to generate a graph-based representation of a risk tensor, the predictive data analysis computing entity 106 may embed/convert/transform data items in the given risk tensor into a graph (e.g., a multiplex graph) representation (e.g., by converting the risk tensor into a graph embedding, for example by using a Node2Vec feature embedding routine). For example, given a genomic risk tensor, if suitable genomic networks for any diseases under consideration are available from the Kyoto Encyclopedia of Genes and Genomes (KEGG) resource (e.g. for non-small cell lung cancer, the genomic pathway, available online at https://www.genome.jp/kegg-bin/show_pathway?hsa05223), then the pathway may be converted to a graph representation in order to generate a tensor-based graph feature embedding for the genomic risk tensor. In some embodiments, when a tensor-based graph feature embedding is used to generate a trained tensor-based graph processing machine learning model (e.g., by using ground-truth tensor-based graph feature embeddings as inputs during the training of a tensor-based graph processing machine learning model), the tensor-based graph feature embedding is referred to as a ground-truth tensor-based graph feature embedding. In some embodiments, when a tensor-based graph feature embedding is used to generate inferred hybrid risk scores that are in turn used to generate a hybrid risk score generation machine learning model, the tensor-based graph feature embedding is referred to as a prior tensor-based graph feature embedding.

At step/operation 804, the predictive data analysis computing entity 106 generates the trained tensor-based graph processing machine learning model based at least in part on the set of ground-truth tensor-based graph feature embeddings for each ground-truth prediction input data object of the set of ground-truth prediction input data objects. In some embodiments, to perform step/operation 804, the predictive data analysis computing entity 106 trains a Graph Neural Network Deep Learning model (GNN-DL) for each ground-truth risk tensor based at least in part on the data in that particular ground-truth risk tensor (e.g., as described by the ground-truth tensor-based graph feature embedding for the particular ground-truth risk score). In some embodiments, the specific type of deep learning models may be determined based at least in part on context and representation of the genomic information in the knowledge graph. The algorithm for performing inference in the graph may also be determined by context, such that its particular inductive bias is deemed to be a reasonable match to the data within each particular ground-truth risk tensor.

In some embodiments, to generate a graph-based machine learning model that is configured to process genomic data to generate a per-model machine learning output, depending on the types of genomic data in the genomic risk tensor and the availability of an algorithm to accommodate variants more complex than single-nucleotide polymorphisms (SNPs), the predictive data analysis computing entity 106 begins with an initial equation typical of a polygenic risk score (e.g., an equation characterized by a weighted sum of risk alleles) with additional terms for other variants derived from “best guess” terms in the initial equation for the PRS for the disease(s) under consideration. For each of the remaining types of predictive input feature set data, the predictive data analysis computing entity 106 may utilize a suitable existing risk model (or clinical prediction model) that is reasonably applicable to the data with that specific risk tensor and to the disease(s) in question. These may be the starting (candidate) equations for that particular GNN-DL model, and may be used to bootstrap the overall risk score for the disease under consideration. A summary of exemplary clinical prediction models may be found here: https://www.bmj.com/content/bmj/365/bmj.1737.full.pdf.

In some embodiments, as part of step/operation 804, the predictive data analysis computing entity 106 may partition the existing data to create a hold-out data set for positive cases of the disease(s) under consideration. The positive class may be associated with prediction input data objects that are related to the patients and that have a confirmed diagnosis for the disease(s) under consideration. The training of each model, on a specific risk tensor, may be performed in an end-to-end manner.

Returning to FIG. 7, at step/operation 702, the predictive data analysis computing entity 106 generates the hybrid risk score generation machine learning model using the trained tensor-based graph processing machine learning model. In some embodiments, the predictive data analysis computing entity 106 generates a model that relates a set of prior tensor-based graph feature embeddings for a set of prior prediction input data objects to a set of inferred hybrid risk scores for the set of prior prediction input data objects as generated by the hybrid risk score generation machine learning model, and then generates the hybrid risk score generation machine learning model based at least in part on the noted model.

A hybrid risk score generation machine learning model may be a model that relates one or more tensor-based graph feature embeddings for a prediction input data object to a hybrid risk score for the prediction input data object. For example, the hybrid risk score generation machine learning model may be determined by performing one or more genetic programming operations (e.g., including one or more symbolic regression operations) based at least in part on sets of prior tensor-based graph feature embeddings for a set of prior prediction input data objects and a set of corresponding inferred hybrid risk scores for the set of prior prediction input data objects, where an inferred hybrid risk score for a prior prediction input data object may be determined by processing the set of prior tensor-based graph feature embeddings for the prior prediction input data object using a trained tensor-based graph processing machine learning model, and where the set of prior tensor-based graph feature embeddings for a prior prediction input data object may be supplied as input variables and/or as regressor variables for the one or more genetic programming operations performed to generate the hybrid risk score generation machine learning model. The hybrid risk score generation machine learning model may be configured to process, as inputs, a set of tensor-based graph feature embeddings and generate, as an output, a hybrid risk score, where each tensor-based graph feature embedding may be a vector, and where each hybrid risk score may be an atomic value or a vector.

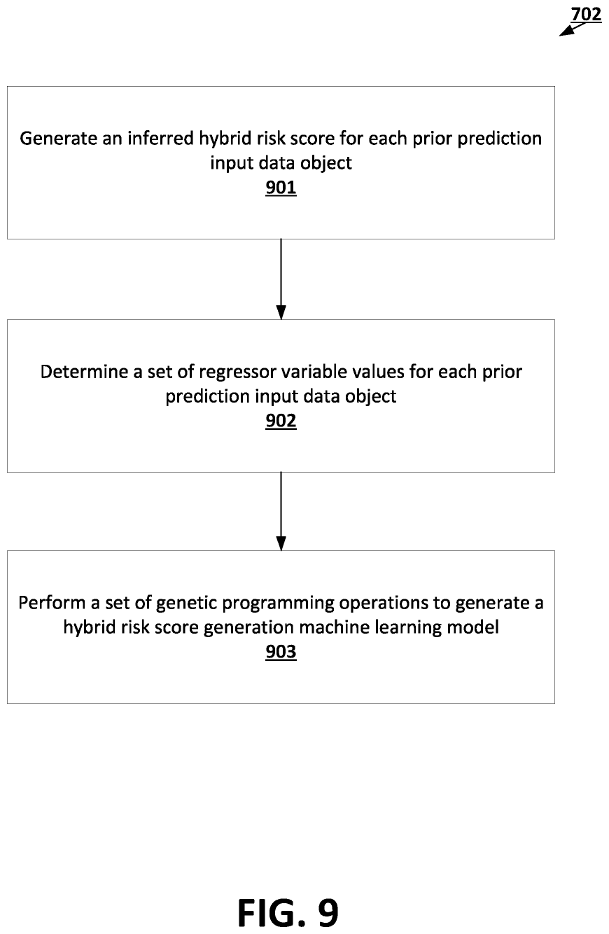

In some embodiments, step/operation 702 may be performed in accordance with the process that is depicted in FIG. 9. The process that is depicted at step/operation 901, when the predictive data analysis computing entity 106 generates an inferred hybrid risk score for each prior prediction input data object of a set of prior prediction input data objects by processing a set of prior tensor-based graph feature embeddings for the prior prediction input data object using the trained tensor-based graph processing machine learning model in order to generate the inferred hybrid risk score for the prior prediction input data object. In some embodiments, as part of step/operation 901, the predictive data analysis computing entity 106: (i) for each of the graph-based machine learning models associated with the tensor-based graph processing machine learning model, partitions ground-truth tensor-based graph feature embedding sets for validation and/or hyper-parameter tuning, and (ii) trains each of the graph-based machine learning models associated with the tensor-based graph processing machine learning model using a partitioned subset of the ground-truth tensor-based graph feature embedding sets that is reserved for training of the tensor-based graph processing machine learning model.

An inferred hybrid risk score may be a risk score that is generated by a trained tensor-based graph processing machine learning model by processing a set of tensor-based graph feature embeddings for a corresponding prediction input data object. For example, the inferred hybrid risk score for a particular prediction input data object may be generated by processing (using a trained tensor-based graph processing machine learning model) the genomic tensor-based graph feature embedding for the particular prediction input data object as determined based at least in part on the genomic risk tensor for the particular prediction input data object, the clinical tensor-based graph feature embedding for the particular prediction input data object as determined based at least in part on the clinical risk tensor for the particular prediction input data object, and the behavioral tensor-based graph feature embedding for the particular prediction input data object based at least in part on the behavioral risk tensor for the particular prediction input data object. The inferred hybrid risk score may be a vector.

At step/operation 902, the predictive data analysis computing entity 106 determines a set of regressor variable values for each prior prediction input data object of a set of prior prediction input data objects based at least in part on the set of prior tensor-based graph feature embeddings for the prior prediction input data object. In some embodiments, using the initial candidate equations for the per-model machine learning output associated with each individual tensor-based graph feature embedding, the predictive data analysis computing entity 106 determines the regressor variables. In some embodiments, the regressor variables are determined based at least in part on techniques for determining separate and interpretable internal functions as described in Crammer et al., Discovering Symbolic Models from Deep Learning with Inductive Biases, arXiv:2006.11287v2, available online at https://arxiv.org/pdf/2006.11287.pdf (2020). In some embodiments, a priori, the predictive data analysis computing entity 106 over-estimates the number of regressor variables so that the algorithm can reduce the parameter space of the hybrid risk score generation machine learning model. In some embodiments, a regressor variable may be any feature or feature-engineered variable that is used in a predictive model as an input to the predictive model.

At step/operation 903, the predictive data analysis computing entity 106 performs a set of genetic programming operations on each prior prediction input data object of a set of prior prediction input data objects to generate the hybrid risk score generation machine learning model. In some embodiments, at step/operation 903, using the initial clinical prediction models and weighted sum of risk alleles (in the case of the genomic risk tensor) or existing outline clinical risk model for the disease(s) in question, the predictive data analysis computing entity performs symbolic regression on each tensor-based graph feature embedding to generate the simplest and most accurate risk model for that tensor-based graph feature embedding. In some embodiments, the genetic programming operations enable a refinement method between the individual graph-based machine learning models of the trained tensor-based graph processing machine learning model. The outputs of the genetic programming operations may be in the form of a graph where the nodes represent mathematical building blocks and edges represent parameters, coefficients and/or system variables. In some embodiments, the one or more genetic programming operations comprise one or more symbolic regression operations, such as one or more symbolic regression operations performed in accordance with the techniques disclosed in Schmidt et al., Symbolic Regression of Implicit Equations, in Genetic programming Theory and Practice VII at 73-85 available online at https://link.springer.com/chapter/10.1007/978-1-4419-1626-6_5 (2009).

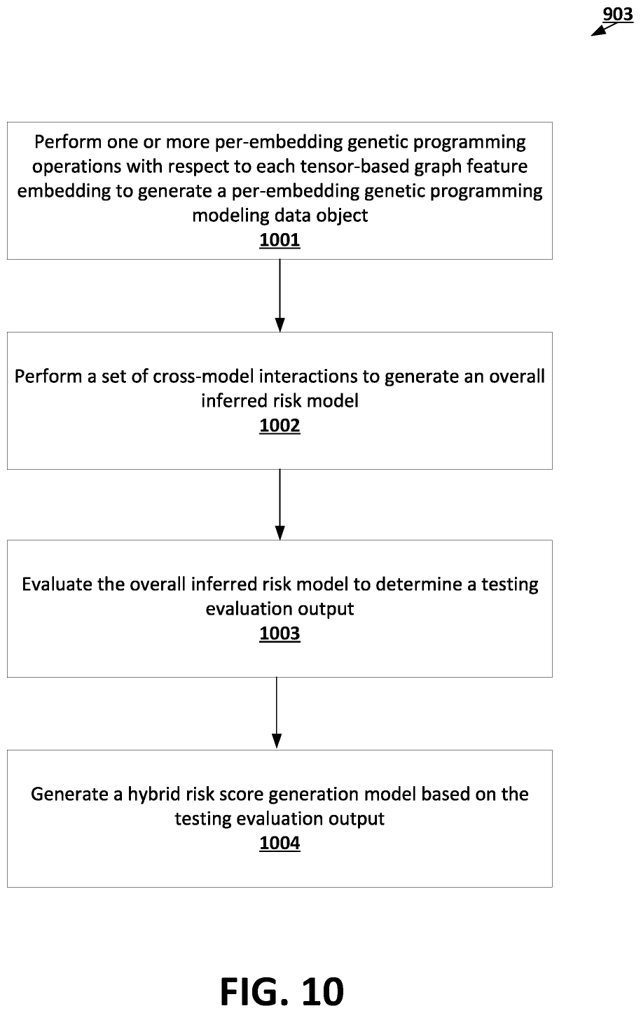

In some embodiments, step/operation 903 may be performed using one or more modeling generation epochs, where performing operations corresponding to a particular model generation epoch may be performed in accordance with the process that is depicted in FIG. 10. The process that is depicted at FIG. 10 begins at step/operation 1001, when the predictive data analysis computing entity 106 performs one or more per-embedding genetic programming operations with respect to each prior tensor-based graph feature embedding for the prior prediction input data object to generate a per-embedding genetic programming modeling data object for the prior tensor-based graph feature embedding. The per-embedding genetic programming modeling data object for a prior tensor-based graph feature embedding for a prediction input data object may be a model that relates the prior tensor-based graph feature embedding to an inferred hybrid risk score for the prediction input data object.

At step/operation 1002, the predictive data analysis computing entity 106 performs a set of cross-model interactions across each per-embedding genetic programming modeling data object for a prior tensor-based graph feature embedding to generate an overall inferred risk model for the model generation epoch. In some embodiments, at each model generation epoch, the predictive data analysis computing entity 106 performs cross-tensor interaction across the per-embedding genetic programming modeling data objects to generate an overall inferred risk model for the model generation epoch. In some embodiments, step/operation 1002 may be performed to optimize the final combined risk equation for the disease(s) under consideration. In some embodiments, the output from the cross-tensor symbolic regression is a candidate equation representing the overall risk for the disease(s) under consideration.

At step/operation 1003, the predictive data analysis computing entity 106 evaluates the overall inferred risk model for the model generation epoch using test data to determine a testing evaluation output for the overall inferred risk model for the model generation epoch. In some embodiments, the predictive data analysis computing entity 106 tests the overall inferred risk model using the hold-out test data. In some embodiments, the final overall risk model needs to meet a pre-determined threshold for accuracy, otherwise the predictive data analysis computing entity 106 produces a warning. In some embodiments, providing additional data, utilizing additional parameters, utilizing other embedding methods, performing feature engineering, and changing model architectures to different types of graph-based machine learning models would all be considered and the entire process re-run until the accuracy threshold is met on hold-out data reserved for testing.

At step/operation 1004, the predictive data analysis computing entity 106 determines the hybrid risk score generation machine learning model based at least in part on the testing evaluation output. If the testing evaluation output describes that the overall inferred risk model meets accuracy requirements, the predictive data analysis computing entity 106 may adopt the overall inferred risk as the hybrid risk score generation machine learning model. However, if the testing evaluation output describes that the overall inferred risk model fails to meet accuracy requirements, a new model generation epoch may be performed. In some embodiments, if the testing evaluation output describes that the overall inferred risk model fails to meet accuracy requirements, actionable outputs are used to refine the next pass through the data once the adjustments have been made (roughly analogous to the back-propagation step in a deep neural network, and perhaps using a lifelong machine learning approach or an incremental learning approach). The hybrid risk score generation machine learning model may then be assessed for simplicity, by ensuring that repeated similar terms are necessary as the model loses accuracy when they are combined.

In some embodiments, the predictive data analysis computing entity 106 enables lifelong machine learning capability for the hybrid risk score, so that it will continue to be updated from “real-world” feedback of its results, e.g., if it predicted that a patient with certain clinical, genomic, behavioral characteristics would be at risk of a certain condition through its generated risk equation and the inferred hybrid risk score was incorrect, this information will be fed back in to the solution (analogously to the back-prop step in a typical deep neural network) so that it can learn from its results and improve over time. In some embodiments, once all accuracy metrics are met, the hybrid risk score generation machine learning model is then ready for use in a clinical trial to determine its accuracy and efficacy in real-world clinical scenarios on diverse patient groups. If the risk equation performs sufficiently in the real-world, it is deemed ready to be accepted for general clinical use in a clinical decision support solution.

In some embodiments, once generated, the hybrid risk score generation machine learning model may be used to generate a hybrid risk score for a prediction input data object (e.g., based at least in part on tensor-based graph feature embeddings for the prediction input data object). In some embodiments, generating a hybrid risk score for a prediction input data object includes processing a plurality of graph feature embedding data objects for the prediction input data object using a hybrid risk score generation machine learning model to generate the hybrid risk score, wherein: (i) the hybrid risk score generation machine learning model is generated using a set of genetic programming operations, (ii) the set of genetic programming operations are performed based at least in part on a set of inferred hybrid risk scores for a set of prior prediction input data objects, and (iii) each inferred hybrid risk score of the set of inferred hybrid risk scores is generated by processing a plurality of prior graph feature embedding data objects for a corresponding prior prediction input data object of the set of prediction input data objects using a tensor-based graph processing machine learning model.

In some embodiments, the tensor-based graph processing machine learning model comprises a plurality of graph-based machine learning models and an ensemble machine learning model. In some embodiments, each graph-based machine learning model of the plurality of graph-based machine learning models is configured to process a prior graph feature embedding data object of the plurality of prior graph feature embedding data objects for a prior prediction input data object of the set of prediction input data objects to generate an inferred hybrid risk score of the set of inferred hybrid risk scores for the prior prediction input data object. In some embodiments, the plurality of graph-based machine learning models comprises one or more graph convolutional neural network machine learning models. In some embodiments, the plurality of graph feature embedding data objects comprises a genomic graph feature embedding data object. In some embodiments, the plurality of graph feature embedding data objects comprises a behavioral graph feature embedding data object. In some embodiments, the plurality of graph feature embedding data objects comprises a clinical graph feature embedding data object. In some embodiments, the set of genetic programming operations comprises a set of symbolic regression operations.

In some embodiments, once generated, the hybrid risk score may be used by the predictive data analysis computing entity 106 to perform one or more prediction-based actions. Examples of prediction-based actions include: generating user interface data for one or more prediction output user interfaces and providing the user interface data to one or more client computing entities 102, displaying one or more prediction output user interfaces to an end user, generating notification data for one or more notification user interfaces and providing the notification data to one or more client computing entities 102, presenting one or more electronically-generated notifications to an end user, and/or the like.

Accordingly, as described in this Subsection, various embodiments of the present invention improve the computational efficiency of performing risk score generation predictive data analysis by describing reliable hybrid risk score generation machine learning models that are trained based at least in part on performing genetic programming operations on the inferred outputs generated by another machine learning model, such as the inferred outputs generated by a hybrid tensor-based graph processing machine learning model. The hybrid risk score generation machine learning models described and enabled by various embodiments of the present invention often require relatively little computational resources (including processing resources and memory resources) to execute. This is because genetic programming operations (e.g., symbolic regression operations) are able to infer often non-complex algebraic relationships between inputs to the hybrid risk score generation machine learning models, a feature that both reduces the runtime cost of performing predictive inferences using the noted inferred hybrid risk score generation machine learning models and improves the need for storing complex configuration data for the inferred hybrid risk score generation machine learning models in order to enable performing predictive inferences using the hybrid risk score generation machine learning models. In this way, various embodiments of the present invention improve both computational efficiency and storage-wise efficiency of performing risk score generation predictive data analysis and make important technical contributions to the field of predictive data analysis in relation to machine learning techniques for generating risk scores. While various embodiments of the present invention discuss performing a set of genetic programming operations, a person of ordinary skill in the relevant technology will recognize that operations of any evolutionary optimization computational method may be used.

Moreover, various embodiments of the present invention improve interpretability of performing risk score generation machine learning models. The inferred hybrid risk score generation machine learning models described and enabled by various embodiments of the present invention describe interpretable relationships between regressor variables, a feature that, in turn, enables a predictive data analysis system to generate explanatory metadata for a generated hybrid risk score. In this way, various embodiments of the present invention improve interpretability of performing risk score generation predictive data analysis and make important technical contributions to the field of predictive data analysis in relation to machine learning techniques for generating risk scores.

Accordingly, as discussed in greater detail above, various embodiments of the present invention introduce techniques that improve the training speed of a graph processing machine learning framework given a constant/target predictive accuracy by using a model deficiency data object that is generated for the graph processing machine learning framework using holistic graph links inferred by a graph representation machine learning model. The combination of the noted components enables the proposed graph processing machine learning framework to generate more accurate graph-based predictions, which in turn increases the training speed of the proposed graph set processing machine learning framework given a constant predictive accuracy. It is well-understood in the relevant art that there is typically a tradeoff between predictive accuracy and training speed, such that it is trivial to improve training speed by reducing predictive accuracy, and thus the real challenge is to improve training speed without sacrificing predictive accuracy through innovative model architectures. See, e.g., Sun et al., Feature-Frequency—Adaptive On-line Training for Fast and Accurate Natural Language Processing in Computational Linguistic 563 at Abst. (“Typically, we need to make a tradeoff between speed and accuracy. It is trivial to improve the training speed via sacrificing accuracy or to improve the accuracy via sacrificing speed. Nevertheless, it is nontrivial to improve the training speed and the accuracy at the same time”). Accordingly, techniques that improve predictive accuracy without harming training speed, such as various techniques described herein, enable improving training speed given a constant predictive accuracy. Therefore, by improving accuracy of performing graph-based machine learning predictions, various embodiments of the present invention improve the training speed of graph processing machine learning frameworks given a constant/target predictive accuracy.

### VI. Conclusion

Many modifications and other embodiments will come to mind to one skilled in the art to which this disclosure pertains having the benefit of the teachings presented in the foregoing descriptions and the associated drawings. Therefore, it is to be understood that the disclosure is not to be limited to the specific embodiments disclosed and that modifications and other embodiments are intended to be included within the scope of the appended claims. Although specific terms are employed herein, they are used in a generic and descriptive sense only and not for purposes of limitation.

1. A computer-implemented method for generating a model deficiency data object for a tensor-based graph processing machine learning model that is associated with a risk category, the computer-implemented method comprising:
identifying, using one or more processors, a positive input set that is associated with the risk category, wherein:
the positive input set comprises a plurality of prediction input data objects that are associated with an affirmative label for the risk category, and
each prediction input data object in the positive input set is associated with: (i) a prediction input feature set, (ii) a plurality of risk tensors each generated based at least in part on a categorical subset of the prediction input feature set for the prediction input data object that is associated with an input category of a plurality of input categories, and (iii) a plurality of tensor-based graph representations generated based at least in part on the plurality of risk tensors for the prediction input data object;
identifying, using the one or more processors, a tensor-based graph representation set for the positive input set, wherein: (i) the tensor-based graph representation set comprises, for each prediction input data object in the positive input set, the plurality of tensor-based graph representations for the prediction input data object, and (ii) the tensor-based graph representation set describes a group of tensor-based graph links;
generating, using the one or more processors and a graph representation machine learning model, and based at least in part on each prediction input feature set, a group of holistic graph links;
generating, using the one or more processors and based at least in part on the group of tensor-based graph links and the group of holistic graph links, the model deficiency data object; and
performing, using the one or more processors, one or more prediction-based actions based at least in part on the model deficiency data object.

2. The computer-implemented method of claim 1, wherein, for a given prediction input data object, the tensor-based graph processing machine learning model is configured to:
for each tensor-based graph representation that is associated with the given prediction input data object, generate a tensor-based graph feature embedding that is associated with a respective input category of the risk tensor that is used to generate the tensor-based graph representation, and
generate an inferred hybrid risk score for the given prediction input data object based at least in part on each tensor-based graph feature embedding that is associated with the given prediction input data object.

3. The computer-implemented method of claim 2, wherein:
the tensor-based graph processing machine learning model comprises a plurality of graph-based machine learning models each associated with a respective input category and an ensemble machine learning model, and
generating the inferred hybrid risk score for the given prediction input data object comprises: (i) for each input category, generating, using the graph-based machine learning model and based at least in part on the tensor-based graph feature embedding for the input category, a categorical tensor-based graph feature embedding, and (ii) generating, using the ensemble model and based at least in part on each categorical tensor-based graph feature embedding, the inferred hybrid risk score.

4. The computer-implemented method of claim 2, wherein:
for each prior prediction input data object of a plurality of prediction input data objects, the plurality of tensor-based graph feature embeddings for the prior prediction input data object are generated by the tensor-based graph processing machine learning model to generate the inferred hybrid risk score for the prior prediction input data object,
a hybrid risk score generation machine learning model is generated using one or more genetic programming operations, and
the one or more genetic programming operations are configured to, for each prior prediction input data object, relate the plurality of tensor-based graph feature embeddings for the prior prediction input data object to the inferred hybrid risk score for the prior prediction input data object.

5. The computer-implemented method of claim 4, wherein the one or more genetic programming operations comprise one or more symbolic regression operations that are configured to generate one or more refined regressor variables for the hybrid risk score generation machine learning model and one or more refined input variables for the hybrid risk score generation machine learning model.

6. The computer-implemented method of claim 1, wherein generating the model deficiency data object comprises:
generating one or more deficiency graph links that are in the group of holistic graph links but are not in the group of tensor-based graph links; and
generating the model deficiency data object based at least in part on the one or more deficiency graph links.

7. The computer-implemented method of claim 6, wherein the model deficiency data object comprises a selected subset of the one or more deficiency graph links that is generated based at least in part on: (i) an immutability score for each deficiency graph link, (ii) an actionability score for each deficiency graph link, and (iii) a prevalence score for each deficiency graph link.

8. An apparatus for generating a model deficiency data object for a tensor-based graph processing machine learning model that is associated with a risk category, the apparatus comprising at least one processor and at least one memory including program code, the at least one memory and the program code configured to, with the at least one processor, cause the apparatus to at least:
identify a positive input set that is associated with the risk category, wherein:
the positive input set comprises a plurality of prediction input data objects that are associated with an affirmative label for the risk category, and
each prediction input data object in the positive input set is associated with: (i) a prediction input feature set, (ii) a plurality of risk tensors each generated based at least in part on a categorical subset of the prediction input feature set for the prediction input data object that is associated with an input category of a plurality of input categories, and (iii) a plurality of tensor-based graph representations generated based at least in part on the plurality of risk tensors for the prediction input data object;
identify a tensor-based graph representation set for the positive input set, wherein: (i) the tensor-based graph representation set comprises, for each prediction input data object in the positive input set, the plurality of tensor-based graph representations for the prediction input data object, and (ii) the tensor-based graph representation set describes a group of tensor-based graph links;
generate, using a graph representation machine learning model, and based at least in part on each prediction input feature set, a group of holistic graph links;
generate, based at least in part on the group of tensor-based graph links and the group of holistic graph links, the model deficiency data object; and
perform one or more prediction-based actions based at least in part on the model deficiency data object.

9. The apparatus of claim 8, wherein, for a given prediction input data object, the tensor-based graph processing machine learning model is configured to:
for each tensor-based graph representation that is associated with the given prediction input data object, generate a tensor-based graph feature embedding that is associated with a respective input category of the risk tensor that is used to generate the tensor-based graph representation, and
generate an inferred hybrid risk score for the given prediction input data object based at least in part on each tensor-based graph feature embedding that is associated with the given prediction input data object.

10. The apparatus method of claim 9, wherein:
the tensor-based graph processing machine learning model comprises a plurality of graph-based machine learning models each associated with a respective input category and an ensemble machine learning model, and
generating the inferred hybrid risk score for the given prediction input data object comprises: (i) for each input category, generating, using the graph-based machine learning model and based at least in part on the tensor-based graph feature embedding for the input category, a categorical tensor-based graph feature embedding, and (ii) generating, using the ensemble model and based at least in part on each categorical tensor-based graph feature embedding, the inferred hybrid risk score.

11. The apparatus method of claim 9, wherein:
for each prior prediction input data object of a plurality of prediction input data objects, the plurality of tensor-based graph feature embeddings for the prior prediction input data object are generated by the tensor-based graph processing machine learning model to generate the inferred hybrid risk score for the prior prediction input data object,
a hybrid risk score generation machine learning model is generated using one or more genetic programming operations, and
the one or more genetic programming operations are configured to, for each prior prediction input data object, relate the plurality of tensor-based graph feature embeddings for the prior prediction input data object to the inferred hybrid risk score for the prior prediction input data object.

12. The apparatus method of claim 11, wherein the one or more genetic programming operations comprise one or more symbolic regression operations that are configured to generate one or more refined regressor variables for the hybrid risk score generation machine learning model and one or more refined input variables for the hybrid risk score generation machine learning model.

13. The apparatus method of claim 8, wherein generating the model deficiency data object comprises:
generating one or more deficiency graph links that are in the group of holistic graph links but are not in the group of tensor-based graph links; and
generating the model deficiency data object based at least in part on the one or more deficiency graph links.

14. The apparatus method of claim 13, wherein the model deficiency data object comprises a selected subset of the one or more deficiency graph links that is generated based at least in part on: (i) an immutability score for each deficiency graph link, (ii) an actionability score for each deficiency graph link, and (iii) a prevalence score for each deficiency graph link.

15. A computer program product for generating a model deficiency data object for a tensor-based graph processing machine learning model that is associated with a risk category, the computer program product comprising at least one non-transitory computer-readable storage medium having computer-readable program code portions stored therein, the computer-readable program code portions configured to:
identify a positive input set that is associated with the risk category, wherein:
the positive input set comprises a plurality of prediction input data objects that are associated with an affirmative label for the risk category, and
each prediction input data object in the positive input set is associated with: (i) a prediction input feature set, (ii) a plurality of risk tensors each generated based at least in part on a categorical subset of the prediction input feature set for the prediction input data object that is associated with an input category of a plurality of input categories, and (iii) a plurality of tensor-based graph representations generated based at least in part on the plurality of risk tensors for the prediction input data object;
identify a tensor-based graph representation set for the positive input set, wherein: (i) the tensor-based graph representation set comprises, for each prediction input data object in the positive input set, the plurality of tensor-based graph representations for the prediction input data object, and (ii) the tensor-based graph representation set describes a group of tensor-based graph links;
generate, using a graph representation machine learning model, and based at least in part on each prediction input feature set, a group of holistic graph links;
generate, based at least in part on the group of tensor-based graph links and the group of holistic graph links, the model deficiency data object; and
perform one or more prediction-based actions based at least in part on the model deficiency data object.

16. The computer program product of claim 15, wherein, for a given prediction input data object, the tensor-based graph processing machine learning model is configured to:
for each tensor-based graph representation that is associated with the given prediction input data object, generate a tensor-based graph feature embedding that is associated with a respective input category of the risk tensor that is used to generate the tensor-based graph representation, and
generate an inferred hybrid risk score for the given prediction input data object based at least in part on each tensor-based graph feature embedding that is associated with the given prediction input data object.

17. The computer program product of claim 16, wherein:
the tensor-based graph processing machine learning model comprises a plurality of graph-based machine learning models each associated with a respective input category and an ensemble machine learning model, and
generating the inferred hybrid risk score for the given prediction input data object comprises: (i) for each input category, generating, using the graph-based machine learning model and based at least in part on the tensor-based graph feature embedding for the input category, a categorical tensor-based graph feature embedding, and (ii) generating, using the ensemble model and based at least in part on each categorical tensor-based graph feature embedding, the inferred hybrid risk score.

18. The computer program product of claim 16, wherein:
for each prior prediction input data object of a plurality of prediction input data objects, the plurality of tensor-based graph feature embeddings for the prior prediction input data object are generated by the tensor-based graph processing machine learning model to generate the inferred hybrid risk score for the prior prediction input data object,
a hybrid risk score generation machine learning model is generated using one or more genetic programming operations, and
the one or more genetic programming operations are configured to, for each prior prediction input data object, relate the plurality of tensor-based graph feature embeddings for the prior prediction input data object to the inferred hybrid risk score for the prior prediction input data object.

19. The computer program product of claim 18, wherein the one or more genetic programming operations comprise one or more symbolic regression operations that are configured to generate one or more refined regressor variables for the hybrid risk score generation machine learning model and one or more refined input variables for the hybrid risk score generation machine learning model.

20. The computer program product of claim 15, wherein generating the model deficiency data object comprises:
generating one or more deficiency graph links that are in the group of holistic graph links but are not in the group of tensor-based graph links; and
generating the model deficiency data object based at least in part on the one or more deficiency graph links.
